(19) United States
(12) Patent Application Publication
(10) Pub. No.: US 2022/0218563 A1
(43) Pub. Date: Jul. 14, 2022

(54) MECHANICAL ENERGY THERAPY DEVICE

(71) Applicant: SynDermix AG, Stans (CH)

(72) Inventors: Carlos Roberto CAMOZZI, Basel (CH); Samuel MALZACH, Evilard (CH); Andrew HIRNIAK, Zürich (CH); Mario TRONZA, Basel (CH); Steve KNUCHEL, Lyss (CH); Carole CHAPELAT, Biel (CH)

(21) Appl. No.: 17/596,091

(22) PCT Filed: Jun. 8, 2020
(86) PCT No.: PCT/EP2020/065865

§ 371 Date: Dec. 2, 2021

(30) Foreign Application Priority Data
Jun. 7, 2019 (CH) 00780/19

(51) Int. Cl. A61H 23/02; B06B 1/04

(52) U.S. Cl. CPC A61H 23/0218; B06B 1/045; A61H 2201/5071; A61H 2201/5028; A61H 2205/022; A61H 2201/0153; A61H 2201/0157; A61H 2201/1685

(57) ABSTRACT

The invention relates to devices and methods in the field of mechanical vibrational energy therapy, in particular oscillation stimulation of a subject. A device comprises a housing and the housing comprises a contact surface for being put in contact with the subject; a sensor element configured to detect a contact between the contact surface and the subject and optionally to transform a contact pressure between the contact surface of the device and the subject to which the mechanical vibrational energy is to be applied into a pressure dependent output signal; and a transducer configured to convert an electric input signal into an axial oscillatory motion of a mass, wherein the transducer comprises a coil and a permanent magnet, wherein the mass can be moved relative to the housing, wherein the relative movement of the mass is configured to cause at least the contact surface to vibrate, and wherein the mass comprises the permanent magnet.

A method is in particular a computer-implemented method and comprises a step S3 of detecting a contact between the device as described and the subject and generating an output signal, wherein a characteristic of the output signal is different in case a contact is detected compared to a case in which no contact is detected. The method comprises further a step S5 of comparing the characteristic of the output signal with a pre-set threshold value.

### FIELD

The invention relates to the field of physical energy therapy. On the one hand, it relates to devices and a methods suitable for mechanical energy therapy. In particular, the invention relates to mechanical energy therapy using oscillations (vibrations), in particular vibration therapy such as modulated vibration therapy. Sound-vibrational therapy, therapy by acoustic energy or therapy by ultrasound are examples of (modulated as the case may be) vibration therapies. The invention relates to devices and methods in particular, but not exclusively, suitable for the treatment of paranasal sinuses, for example for the treatment of chronic rhinosinusitis (CRS). On the other hand, the invention relates to key components and methods suitable for physical energy therapy, this means these components and methods are suitable for but not restricted to mechanical energy therapy.

### BACKGROUND

There is currently no available medication that is specifically approved for the treatment of conditions such as paranasal inflammation such as that characterised by conditions such as CRS. Indeed, the efficacy and safety of medications prescribed by ENT specialists is limited, while surgical procedures for CRS that are invasive or minimally invasive are associated with limited efficacy, safety risks and/or patient aversion. Hence, there is a high unmet medical need for treatment alternatives that are effective, safe, non-invasive and with a fast onset of action to alleviate the life-disrupting symptoms of conditions like CRS.

The use of devices that are suitable for applying physical and/or vibrational energy to a human or animal body is known in medical applications.

For example, WO 2011/159317 A1 discloses a pain abatement device that provides for multiple sensory inputs, wherein the multiple sensory inputs are generated by temperature, a tactile input and vibration by utilizing multiple small vibratory motors.

US 2012/0253236 A1 discloses wearable devices for externally delivering therapeutic stimulation to improve health, condition and performance. The stimulation is done via vibration, tones, audio or electrical pulse, light or other sources. In embodiments, the device comprises a regular or vibration speaker or a vibrating component with a motor.

US 2003/0172939 A1 discloses a method and a device to relieve discomfort by attaching a vibration generating means to hard tissue of the patient's head and by applying vibrations at a subsonic frequency.

US 2008/0200848 A1 discloses a method and a device for treating nasal congestion and/or relieving sinusitis symptoms, in particular by combining vibrational stimulation and a stream of fluid forced towards the patient's respiration tracks.

US 2013/0253387 A1 discloses systems and methods for treating an occluded area in a body or for reducing pathologic material in the body, for example. Therefore, vibratory energy is applied to pathologic material in a treatment area of the body. The vibratory energy is provided to the treatment area by use of a piezoelectric transducer and an effector, wherein the effector can be designed to reach into the occluded area or to be positioned on a forehead or another external body portion.

WO 2010/113046 A1 discloses a device for the ventilation of nitric oxide in the paranasal sinuses and to suppress disorders of the upper respiratory tract. The device comprises a vibration generator, a vibration transmitter in mechanical/physical contact with the vibration generator, and a control unit. The vibration generator contains an electric motor and an eccentric wheel. Control unit, vibration generator and vibration transmitter are designed to allow for a fast revolution changes in a given frequency range.

EP 3 446 745 A1 describes a device for applying ultrasound as well as electromagnetic radiation to the skin. The device has transducers comprised in a treatment head which provide for two types of stimulation—vibrational stimulation and electrical stimulation. The device is also able to provide heat treatment. The device further comprises a detector which is a sensor able to detect contact with the skin. The stated purpose of this device is for cosmetic applications on the skin.

US 2015/165238 A1 describes a treatment device having an energy source and a rolling member so that treatment can be provided at multiple locations through movement of the rolling device. The energy source is an ultrasound transducer. The device further comprises a contact sensor which can measure capacitance of a surface.

KR 2017/0111945 A discloses apparatus comprising a product recognition unit for recognizing information of a skin-applying type product for massage, and a control unit for generating a control signal for the massage mode according to information of the skin and a massage unit operating in the massage mode according to the control signal of the controller.

US 2014/194794 A1 describes a massager that includes a massager head with a capacitive sensor. A controller uses the capacitive sensor to sense capacitance changes that indicate a human body is in close proximity or in contact with the massager head.

US 2017/087379 discloses devices and methods for light therapy of acne. The device can comprise a capacitor touch sensor and a micro-vibration motor.

US 2015/005750 A1 discloses a device which is used to treat eyelids, meibomian glands, ducts, and surrounding tissue primarily by light. However the type of energy emitted by a transducer can vary from light to acoustic, radio frequency, electrical, magnetic, electromagnetic, vibrational, infrared or ultrasonic energy. The device can further comprise a safety sensor to monitor the proximity between the energy transmission surface and the surface of the eyelid.

It is an object of the invention to overcome drawbacks of state-of-the-art devices and methods, for example at least one of the drawbacks related to the treatment parameters used, user-friendliness, support to the user, and improve monitoring of the treatment, especially in real time.

For example, it is an object of the invention to provide a treatment device and a method having an increased percentage of successful treatments and reduced undesirable or unexpected adverse effects.

For example, it is an object of the invention to improve user-friendliness.

It is a further object of the invention to provide key components of such a treatment device.

It is a further object of the invention to provide a device, key components for such a device and a method suitable for the treatment of CRS by (external) vibration therapy, in particular modulated vibration therapy, wherein the device and method overcome drawbacks of state-of-the-art devices and methods used for the treatment of chronic rhinosinusitis (CRS).

At least one of these objects is achieved by the devices and methods according to the claims.

### SUMMARY OF THE INVENTION

The invention concerns different aspects that alone or in combination achieve at least one of these objects.

In principle, each of the aspects discussed in the following can be considered as a separate invention and has the potential to be the subject-matter of an independent claim. However, the aspects are interlinked, and any combination of aspects is conceivable and has synergetic effects, for example for achieving at least one object in a better manner and/or for achieving a plurality of objects mentioned above.

In particular, the first and second aspects form a group of inventions linked by a sensor element configured to detect a contact between the contact surface of the device and the subject to be stimulated and the generation of an output signal, wherein a characteristic of the output signal is different in case a contact is detected compared to a case in which no contact is detected.

A first aspect concerns a device for applying physical energy to a subject to be stimulated, wherein the device comprises a sensor element configured to detect a contact between the contact surface and the subject and optionally to transform a contact pressure between the contact surface of the device and the subject to which the mechanical energy is to be applied into a pressure dependent output signal.

The first aspect relates further to a related method for treating a subject with mechanical energy, in particular with oscillations (vibrations).

A second aspect concerns a computer-implemented method for supporting the user in a long-lasting treatment, wherein the treatment comprises a step of bringing a device in contact with a subject to be treated and maintaining the device in this contact for some time before removing the device again. The treatment can be long lasting because it comprises maintaining the contact between the device and the subject for a longer time and/or because the treatment comprises bringing the device at a plurality of positions in contact to the subject, for example.

The method comprises a step of detecting a contact between the device and the subject and optionally a step of measuring a contact pressure between the device and the subject.

The second aspect relates further to a related method for treating a subject with mechanical energy, in particular with oscillations (vibrations).

A third aspect concerns a device for applying mechanical energy, in particular oscillations, to a subject to be stimulated, wherein the device comprises a transducer, in particular a vibration generator, that comprises a coil, in particular a coil as disclosed in the following. A coil as disclosed in the following is sometimes called a voice coil.

A fourth aspect concerns a device for applying physical, in particular mechanical, energy to a subject to be stimulated, wherein the device comprises a movable device head that can be moved to a plurality of positions relative to a device body.

In particular the sensor element as disclosed in the following and the transducer as disclosed in the following are key components of the device that can be used in various technical fields and devices. Hence, the invention is not restricted to devices for physical energy therapy but also relates to the sensor element and the transducer itself. In other words, the sensor element and the transducer can be considered as independent (separate) inventions.

The invention also concerns devices equipped for carrying out any method according to any aspect and any embodiment described in the present text and any combination thereof.

The invention also concerns methods comprising the steps for operating the device according to any aspect and any embodiment described in the present text and any combination thereof.

### BRIEF DESCRIPTION OF THE DRAWINGS

The invention is further illustrated with reference to the accompanying drawings which schematically show:

FIG. 1 an exterior view of an exemplary embodiment of a device;

FIG. 2 an external view of a further exemplary embodiment of a device;

FIG. 3 an external view of yet a further exemplary embodiment of a device;

FIG. 4 an exploded view of device shown in FIG. 1;

FIG. 5 an exploded view of an exemplary embodiment of the device head shown in FIG. 1;

FIG. 6 an exploded view of a further exemplary embodiment of a device head;

FIGS. 7-9 schematics that visualize the operating principle of an exemplary sensor element;

FIG. 10 a sectional view of the device head of FIG. 5;

FIG. 11 an exploded view of an exemplary embodiment of a transducer;

FIG. 12 a sectional view of the transducer of FIG. 11;

FIG. 13 a detail view of an actuation region of the transducer shown in FIG. 11;

FIG. 14 a detail view of an alternative embodiment of the actuation region;

FIG. 15 a flow chart of a computer-implemented method for supporting a user in a mechanical energy treatment;

FIG. 16 a flow chart of a computer-implemented method for supporting a user in a mechanical energy treatment, wherein the method comprises a determination of a contact quality;

FIG. 17 a flow chart of a computer-implemented method for supporting a user in a mechanical energy treatment, wherein the method comprises a determination of a treatment regularity;

FIG. 18 a flow chart of a computer-implemented method for supporting a user in a mechanical energy treatment, wherein the method comprises a determination of treatment completeness;

FIG. 19 a flow chart of a computer-implemented method for supporting a user in a mechanical energy treatment, wherein the method comprises a determination of a treatment quality;

FIGS. 20-23 CRS treatment as an application example;

FIG. 24 shows a schematic block diagram of functional components comprised within an exemplary embodiment of a device; and

FIG. 25 shows (a) graphs of signal amplitude versus frequency (top) and frequency versus time (bottom) for device of an embodiment of the invention, whilst shown in (b) are graphs of signal amplitude versus frequency (top) and frequency versus time (bottom) for control device.

### DETAILED DESCRIPTION OF THE INVENTION

Unless otherwise defined, all technical and scientific terms used herein have the same meaning as commonly understood by one of ordinary skill in the art to which this invention belongs. Independent of the aspect of the invention and of embodiments thereof, the following terms have the following meaning if not stated explicitly otherwise.

As used herein, the term ‘comprising’ means any of the recited elements are necessarily included and other elements may optionally be included as well. ‘Consisting essentially of’ means any recited elements are necessarily included, elements that would materially affect the basic and novel characteristics of the listed elements are excluded, and other elements may optionally be included. ‘Consisting of’ means that all elements other than those listed are excluded. Embodiments defined by each of these terms are within the scope of this invention.

Physical energy comprises mechanical energy, such as oscillations, but also radiation (such as radiation in the visible (“light”) or infrared wavelength range), temperature and electrical stimulation, for example, wherein the radiation, temperature and electric stimulations used is in a range suitable for cosmetic applications, therapeutic applications and/or applications for well-being.

If the physical energy is mechanical energy, the mechanical energy is provided by oscillations in the embodiments disclosed in the following, this means the mechanical energy is vibrational energy.

A treatment is “long-lasting” if the treatment takes some time, for example more than 5, 10, 15 or 30 s, such as 1 minute or more. The treatment can take some time because a step of the treatment other than any preparation or subsequent step takes some time and/or because a step other than any preparation or subsequent step is repeated a several times.

In the field of physical, in particular mechanical, energy therapy, it is important that the treatment is carried out with treatment parameters each of it being within a range determined by the treatment to be carried out. The treatment parameters comprise operational parameters of the treatment device, such as amplitude, intensity, frequency and treatment time, parameters representative for treatment steps of a treatment comprising a sequence of steps, as well as parameters relevant for the interaction between treatment device and the subject to be treated, such as the site of application (also called “position” in the following) and orientation of the treatment device relative to the subject and the properties of the contact between the treatment device and the subject. The number of treatment sessions in a sequence of treatments, for example the number of treatment sessions within a given period of time, and the time of a treatment in a sequence of treatment sessions are examples of parameters representative for the treatment steps.

The properties of the contact between the treatment device and the subject comprise the contact pressure, the design of a contact area and the physical, in particular mechanical, properties of an element forming the device-side portion of the contact area and the physical, in particular mechanical, properties of the subject-side region forming the contact area of the application site.

The treatment parameters depend on the region to be treated and the effect to be generated. The nasal cavity and para-nasal sinuses are examples of treatment target areas in the head.

Stimulation of cell/tissue activity and/or removal of a secretion, like abnormal mucus or purulent secretion, are examples of different possible therapeutic expected effects of vibrational therapy.

In many cases, the treatment parameters are specifically related to the subject and to the individual subject and/or to the human or animal patient to be treated.

One treatment parameter that is not delivered in the range imposed by the desired application can significantly reduce the expected therapeutic efficacy and/or causes undesirable or unexpected adverse effects.

In principle, any mechanical energy treatment that potentially stimulates a region not intended to be treated bears the risk of generating an undesirable adverse effect. This risk is especially high when the treatment target organ(s) or tissue(s) are in the head and/or the region to be treated is a region of the head and/or the stimulation is applied via an external head portion. For example, this is because the head comprises with the hard tissue forming the skull a structure capable to transmit mechanical energy, but it comprises limited amounts of soft tissue and separation fluids between hard tissue only, both being important for damping of the mechanical energy. Due to this structure of the head, non-optimised treatment parameters can cause undesirable or unexpected adverse effects in the contact area and/or the region to be treated as well as in regions remote from the contact area. Toothache and hearing disorder are examples of undesirable or unexpected adverse effects.

Similar concerns are valid for other subjects such as the hip, the shoulder or the ankle, for example.

The range imposed by the desired application depends on the region to be treated, the effect to be generated and is subject- and patient-specific in many cases, as pointed out above.

State of the art treatment devices and methods consider the influence of the operational parameters on the treatment success and undesirable or unexpected adverse effects to a certain extend only (for example WO 2010/113046 A1) and they nearly ignore the influence of parameters relevant for the interaction between treatment device and the subject or they provide workarounds (for example US 2013/0253387 A1 providing an effector designed to reach into the occluded area).

Further, state of the art treatment devices and methods lack user-friendliness, support to the user during treatment, and monitoring of the treatment.

Handling, of the device, compliance, perception of the treatment, of the therapeutic effect and disease or clinical condition progression are some examples of topics related to user-friendliness.

In many cases, the issue of support during treatment is directly linked to the issue of treatment parameters being in the ranges imposed by the desired application and hence to treatment success and efficacy.

Suitable monitoring of the treatment can be used for feedback to the user during treatment, for example for support during treatment. Monitoring may occur in real time, such as via a mobile or computer application (an ‘app’) that monitors clinical parameters transmitted from the device via mobile telemetry (e.g. bluetooth or over a wireless network).

Alternatively, or in addition, monitoring can be used after treatment or between two treatments of a cycle of treatment, for example indicating additional treatments or proposals for amendments for increasing treatment success.

Alternatively, or in addition, monitoring can be used for an adjustment of the treatment parameters.

Embodiments of the device and method according to the invention are in particular suitable for vibration therapy, in particular modulated vibration therapy, that is applied to an exterior body portion.

In embodiments suitable for modulated vibration therapy, the frequency is modulated at least, for example by applying a sweep as disclosed below.

Vibration therapy is used for several medical applications such as chronic rhinosinusitis (CRS), migraine, chronic wound healing, pain relief, nasal congestion and muscular tension.

There are indications of a potential use of vibration therapy in various further medical applications as pointed out below.

The main advantages of (exterior) vibration therapy over other therapy methods are its non-invasive, drug-free and safe character without significant loss of local applicability if directed vibrations (as provided by the device according to the invention) are used. Further advantages are easy and comfortable applicability if a treatment is carried out with a device according to the invention.

The specific biological, physical and chemical effects caused in a living body by vibration therapy are still being investigated in future trials, but the general effects are discussed in the following. The general effects of vibration therapy comprise vasodilatation, stimulation of cells, and enhancement of secretion clearance (for example by promoting transport and/or (out)flow) among others.

In the following, it is shown on the example of the treatment of chronic rhinosinusitis (CRS) how these effects cause a significant therapeutic effect. The device is in particular configured to cause at least one of these effects and hence to cause said therapeutic effect (as shown in the “application example” given below).

If a device for vibration therapy is applied on the cheekbone for the treatment of CRS (chronic rhinosinusitis), vibrations propagate to the paranasal sinuses like the maxillary sinus and to the nasal cavity and set the paranasal sinuses and the nasal cavity in oscillation.

These oscillations accelerate the transport in the nose of excessive mucus and secretions, for example by mechanically induced transport and/or by increase of the mucociliary clearance, and stimulate the nasal and paranasal epithelium, for example by setting the epithelium in vibration and by vasodilatation. Both accelerated transport and stimulation accelerate the healing process, in particular reduce inflammation, and contribute to an opening of the ostium of the paranasal sinuses. The latter in combination with a vibrating maxillary sinus allows for a promptly release of nitric oxide (NO) from the paranasal sinuses into the nasal cavity. In addition, the vibration of the maxillary sinus presumably promotes NO production. There are indications that a high NO concentration has a protective or even healing effect, said effect being active in the maxillary sinus and nasal cavity due to the given mechanism of action.

In summary, vibration therapy enhances and accelerates the healing process, reduces the pathognomonic symptomatology of CRS (e.g. facial pain, congestion, rhinorrhoea, etc.) and improves the well-being of the patient with CRS both in the short and long-term. In other words, it shows anti-inflammatory, antioedematous and antiallergic effects, promotes normalisation of body defences, and may be used as monotherapy. The method is physiological, and it reduces the number of punctures in maxillary sinusitis, leaves the skin and mucosa intact, and decreases the use of drugs.

The mechanism of action summarized in the preceding paragraphs will be further explored using the device disclosed.

The effect of applying a device as disclosed may be further explored in clinical tests.

Evaluation of at least one of the change in subjective symptoms may be quantified by the German validated disease-specific 20-item Sino-nasal Outcome Test (SNOT-20 GAV), the change in endoscopic appearances, the change in need for surgical intervention, the change in the ability to perform normal activities, overall disease control, acceptability of treatment, overall score SNOT-20, pain score (VAS), and adverse events.

Vibration therapy in general has the potential for treating various medical conditions and reasons for physical uneasiness based on the biological, physical and chemical effects mentioned above and if the applied vibrations have characteristics suitable to cause these effects.

There are indications that vibration therapy increases angiogenesis and granulation tissue formation and reduces neutrophil accumulation and increases macrophage accumulation.

Additionally, it may increase expression of pro-healing growth factors and chemokines (insulin-like growth factor-1 (IGF-1), vascular endothelial growth factor (VEGF) and monocyte chemotactic protein-1) in wounds (Eileen M. et al., 2014; PLoS ONE 9(3)). Vibration exposure may increase gene expression of collagen-1α (3-fold), IL-6 (7-fold), COX-2 (5-fold), and bone-morphogenetic-protein-12 (4-fold) (Thompson W et al., The Orthopaedic Journal of Sports Medicine, 3(5)).

A device according to the first aspect is suitable for applying physical energy to a subject to be stimulated.

In particular, the device can be suitable for applying mechanical energy such as vibration, for example in the acoustic energy range or ultrasound, in particular in the low frequency acoustic or even infrasound energy range to the subject to be stimulated. In other words, the mechanical energy applied can have any frequency disclosed in relation to the device according to the third aspect. In particular, the frequency can be in the range of 1 Hz to 2000 Hz, for example in the range of 20 Hz to 1500 Hz, preferably in the range of 60 Hz to 1300 Hz.

The device comprises a device head and optionally a device body. The device body can be designed for being held by a user.

The device can be a handheld device.

The device can be portable.

The device can be configured for a drug free use.

The device can be configured for a non-invasive use.

The device body can be as described with respect to the third and/or fourth aspect.

The device, in particular the device head, is designed to comprise a surface (called “contact surface” in the following) that can be brought in contact to the subject, for example when the device is held at the device body and when the device is in a state suitable for stimulation of the subject.

The device can be configured for direct contact between the surface and the subject, this means between the surface and the skin of the body portion to which the device is applied, during use. In other words, there is no need for an intermediate element or layer between the surface and the skin. In particular, there is no need for a gel and the like.

It is possible that the device is not in a state suitable for stimulation of the subject because the device comprises a movable device head that can be moved to a plurality of positions relative to the device body as described in relation to the fourth aspect.

The device head can be as described with respect to the third and/or fourth aspect.

The device according to the first aspect comprises further a sensor element that is configured to detect a contact between the contact surface and the subject and to generate a related output signal. A characteristic value (in short “a characteristic”) of the output signal is different in case a contact is detected compared to a case in which no contact is detected.

In other words, the characteristic has a first value if there is no contact between the contact surface and the subject and it has a second value that is different from the first value if there is a contact between the contact surface and the subject.

The sensor element can be arranged in the device head.

The sensor element can comprise any means capable to detect the presence of the subject on the contact surface. For example, the sensor element can comprise means for measuring a current, a voltage (tension) or a resistance, a pressure sensor or a capacitive sensor.

The contact between the contact surface and the subject can be established as soon as the contact surface and the subject touch each other.

However, the contact between the contact surface and the subject is established as soon as the contact surface and the subject touch each other in a manner suitable for applying mechanical energy to the subject. In particular, the characteristic of the output signal has a value indicating a contact only if a certain contact pressure is established at the contact surface and the subject.

In an embodiment, the sensor element is configured further to measure the contact pressure.

In other words, the sensor element is configured to transform the contact pressure between the contact surface and the subject into a pressure dependent output signal, for example a voltage (tension), current or resistance.

The output signal can be the pressure dependent output signal. In this case, the pressure dependent output signal can indicate a contact between the contact surface and the subject as soon as the pressure dependent output signal is larger than a pre-set value.

In an embodiment, the sensor element comprises a capacitive sensor that is configured to detect the subject when being in contact with contact surface.

Optionally, the capacitive sensor can be configured to measure the contact pressure.

The capacitive sensor can be arranged in the device head adjacent to a rear side of an element comprising the contact surface, wherein a front side of the element comprises the contact surface

The capacitive sensor can use projected capacitive touch (PCT) technology.

In a preferred embodiment, the contact surface comprises at least one indentation that is designed to be filled by material of the subject in a pressure dependent manner.

The indentation can be arranged relative to the capacitive sensor in a manner that different filling states of the indentation, this means the occupancy state defined by different amounts of the material of the subject and/or different degrees of filling of the indentation by said material, lead to different pressure dependent output signals of the sensor element.

For example, the indentation is an indentation in the element comprising the contact surface, wherein the indentation expands from the contact surface towards the rear side of the element, wherein the capacitive sensor is arranged adjacent to the rear side of the element.

A shape of the indentation can be adapted to the subject and/or desired treatment. For example, the shape of the indentation can be designed in a manner that the capacitive sensor in combination with the indentation is most sensitive in the pressure range of importance for a specific subject and/or desired treatment.

The contact surface can comprise the indentation and can be part of an interchangeable part of the device. Different interchangeable parts can further distinguish in the shape of the contact surface. In such embodiments, the same device can be used for various subjects and/or treatments by replacing or moulding the interchangeable part to conform to the anatomy of a subject. Hence, by changing the shape of the contact surface of the device and/or the operational range of the unit indentation/capacitive sensor may be extended.

First experiments have shown that a capacitive sensor in combination with an indentation is an embodiment of the sensor element configured to transform a contact pressure between the contact surface and the subject into the pressure dependent output signal that is very promising for the technical field of mechanical energy therapy.

The use of a capacitive sensor, for example in combination with an indentation, has various advantages for use in a mechanical energy treatment device. In particular, it allows the detection of a contact of the device to the subject to be stimulated, wherein the detection is not disturbed, or disturbed to a limited extend only, by factors like light, water, and touching the device at portions different from the contact surface. In other words, the use of a capacitive sensor, for example in combination with the indentation, allows for a detection of the contact that is reliable compared to other detection approaches.

In addition, the use of a capacitive sensor, for example in combination with an indentation, allows for a determination of the contact pressure in a reliable manner.

In an embodiment, the device comprises further a controller that is configured to determine whether the characteristic of the output signal or optionally a characteristic of the pressure dependent output signal is larger than a pre-set value.

The pre-set value can be a threshold value indicative of a contact between the contact surface and the subject. In other words, the controller can be configured to determine whether the contact surface is in contact to the subject.

The pre-set value can represent a minimal threshold contact pressure needed for carrying out a treatment successfully. In other words, the controller can be configured to determine whether the contact pressure between the contact surface and the subject is sufficient to carry out a treatment.

The pre-set value can depend on the desired treatment.

The pre-set value can depend on at least one of the subject and the patient.

In an embodiment, the device, in particular the controller, is configured to prevent a start of a stimulation if the characteristic of the output signal or optionally the characteristic of the pressure dependent output signal is smaller than the pre-set value.

The device, in particular the controller, can be configured to start a stimulation only if the characteristic of the output signal or optionally the characteristic of the pressure dependent output signal is greater than the pre-set value.

The device can be configured to start a stimulation automatically if the characteristic of the output signal or optionally the characteristic of the pressure dependent output signal is greater than the pre-set value.

Usually, a treatment is started by activation (switching on) of the transducer.

The controller can be configured to determine whether the characteristic of the output signal or optionally the characteristic of the pressure dependent output signal is greater than the pre-set value repeatedly during a treatment.

For example, the controller can be configured to check whether there is a contact or a contact sufficient for carrying out a treatment after the start of the treatment. This feature of the controller can be important for a monitoring of the treatment, such as the determination of a parameter that is representative for the quality of the treatment, such as the contact quality and/or the treatment quality discussed below.

In an embodiment, the controller is configured to set a timestamp when a stimulation is started.

The timestamp can be a signal carrying no other information than the information that a stimulation has started.

The timestamp can comprise further information concerning the start of a stimulation, such as the time of the start of the treatment and/or at least one treatment parameter.

In an embodiment, the controller is configured to determine a treatment regularity by comparing a period between two timestamps with a pre-set period.

The pre-set period can be the optimal period between two treatments for a specific treatment.

The pre-set period can vary in a sequence of treatments. For example, it can be smaller at the beginning of the sequence of the treatment and larger at the end of the treatment.

If the treatment concerns sinusitis for example, an effective sequence of treatments can comprise four treatments during a predefined period of time, such as a day, wherein the last (fourth) treatment of the sequence starts between 3 and 5 hours after the first treatment of the sequence and wherein at least two further treatments, in particular the third and fourth treatment, are carrier out for example within 1 to 10 minutes after the first treatment and within 1 to 10 minutes before the last (fourth) treatment, respectively.

In other words, the pre-set period for the first and second treatment can be between 1 and 10 minutes, the pre-set period for the third and fourth treatment can be between 1 and 10 minutes, and the pre-set period between the second and third treatment can be between 3 and 5 hours (between 3 h minus the pre-set period for the first and second treatment and minus the pre-set period for the third and fourth treatment and 5 h minus the pre-set period for the first and second treatment and minus the pre-set period for the third and fourth treatment, to be more precisely).

A consideration of the treatment times, in particular the calculation of the end of a treatment, can be needed in some embodiments.

The determination of the treatment regularity can comprise a comparison of a period between two sequences of treatments with a pre-set period. The controller can be configured to carry out said comparison.

The first and second treatment of the example given above can be considered as a first sequence of treatments and the third and fourth treatment can be considered as a second sequence of treatments. In this exemplary embodiment, it is time between the timestamp of the second treatment and the timestamp of the third treatment minus the treatment time of the second treatment that can be compared with the pre-set period, for example.

In an embodiment, the controller is configured to determine a treatment completeness by comparing a number of timestamps with a pre-set number of treatments, in particular by comparing a number of timestamps set during a period (e.g. a day or a week) in which the overall treatment is planned to take place with a pre-set number of treatments.

For example, the controller can be configured to count the timestamps generated or received and to compare the number of counted timestamps with a pre-set number of treatments.

The pre-set number of timestamps can depend on the desired treatment. In particular, it can be the number needed to complete the desired treatment.

The pre-set number of timestamps can depend on at least one of the subject and the patient.

The controller can be configured to consider in the determination of the treatment completeness an outcome of the determination of a treatment regularity.

The controller can be configured to consider in the determination of the treatment completeness a treatment parameter monitored during treatment and/or a parameter that is representative for the quality of the treatment, such as the contact quality and/or the treatment quality discussed below.

For example, the controller can be configured to ignore a timestamp or to weight a timestamp, for example with a value between 0 (timestamp ignored) and 1 or 2 or 5.

The monitored treatment parameter can be at least one of the treatment time and the number of treatments in a pre-set period of time (e.g. a day or a week), for example.

In an embodiment, the controller is configured to determine whether the characteristic of the output signal or optionally the characteristic of the pressure dependent output signal is greater than the pre-set value several times during a treatment—i.e. periodically or repeatedly. In this embodiment, the controller is configured further to determine a contact quality by setting the number of characteristics greater than the pre-set value in relation to the total number of output signals.

More precisely, the controller is configured to (i) count the total number N_{T} of determinations made, this means the total number of determination whether the characteristic of the output signal (of the pressure dependent output signal as the case may be) is greater than the pre-set value, (ii) to count the total number N_{P} of determination with a positive outcome, this means the total number of determination showing that the characteristic of the output signal (of the pressure dependent output signal as the case may be) is greater than the pre-set value, and (iii) to set these two numbers in relation.

For example, the ratio R_{CQ}=N_{P}/N_{T} can be determined.

The controller can be configured further to set the total number N_{T} of determinations made and the total number N_{P} of determination with a positive outcome, for example the ratio R_{CQ}=N_{P}/N_{T}, in relation to a reference value that is representative for a good, enough or insufficient contact quality during treatment.

For example, the contact quality during a treatment can be considered as good if R_{CQ}>R_{ref}, wherein R_{ref} is close to 1.

The reference value, for example R_{ref}, can be provided by a doctor (practitioner), the supplier of the device or an application (app), for example.

The controller can be further equipped to determine the contact quality of a sequence of treatments, for example the sequence of treatments needed to accomplish a desired treatment of a plurality of treatments carried out in a given period of time (such as a day or a week).

For example, the controller can be configured to determine an average contact quality.

For example, the controller can be configured to determine

$${R}_{avg} = \frac{1}{n} {\sum }_{1}^{n} {R}_{CQ}$$

and to compare R_{avg} with R_{ref}.

In an embodiment, the sensor element is configured to transform the contact pressure between the contact surface and the subject into the pressure dependent output signal and the controller is configured to read out the pressure dependent output signal.

In this context, “the controller is configured to read out” means that the controller is configured to determine the value of the characteristic that is related to the contact pressure.

The controller can be configured to read out the pressure dependent output signal several times during a treatment.

For example, the controller can be configured to read out the pressure dependent output signal periodically, for example with a given frequency.

The controller can be configured to measure or approximate the value of the characteristic that is related to the contact pressure in dependence of time. In other words, the controller can be configured to measure or approximate the time evolution of said value.

In an embodiment, the controller is configured to read out the pressure dependent output signal several times during a treatment and to determine a treatment quality by setting the read out pressure dependent output signals, in particular the value of the characteristic that is related to the contact pressure, in relation to a target value. The target value may be a time-dependent target value.

For example, a good treatment quality is ensured if the read-out pressure dependent output signal is larger than the target value during at least 50% of the treatment time, in particular during at least 60%, at least 70%, at least 80% or at least 90% of the treatment time. In other words, a good treatment quality is ensured if the pressure applied during the treatment time is above a pressure threshold value during at least 50%, at least 60%, at least 70%, at least 80% or at least 90% of the time of the treatment period.

For example, the controller can be configured to integrate (summarize) the time evolution of the value of the characteristic that is related to the contact pressure from starting time to stop time of the treatment.

The controller can be configured further to set the resulting value in relation to the related integral of a target value of the characteristic that is related to the contact pressure or of a target time evolution of said value from starting time to stop time of the treatment.

For example, the controller can be configured to calculate a ratio between the integral of the read out value and the integral of the target value or of the target time evolution.

The controller can be configured further to compare the ratio with a reference value that is representative for a good, enough or insufficient contact quality during treatment.

For example, a ratio lager than 1 can be considered as a good contact quality leading to a good treatment quality.

For example, a ratio between 0.5, 0.6, 0.7, 0.8 or 0.9 and 1 can be considered as a contact quality sufficient for an acceptable treatment quality.

For example, a ratio below the lower limit for a good contact quality or—as the case may be—below the lower limit for a sufficient contact quality can be considered as an insufficient contact quality resulting in an insufficient treatment quality. In particular, a ratio below 0.5 can be considered as insufficient.

The controller can be configured to avoid distortion of the determined treatment quality and/or contact quality by periods of high contact pressure, for example by capping the read-out pressure dependent output signal.

Alternatively to a controller being configured to carry out the calculations discussed above, the device can comprise communication means to a computerized device that is configured to carry out said calculations.

Independent of the embodiment of the device and the aspect, the computerized device with which the device may communicate can be a cell phone or any other computerized device owned by the costumer, for example a tablet or PC.

Alternatively, the computerized device can be a remote computerized device with which the device may communicate directly by comprising means to establish a communication channel to the remote computerized device or with which the device may communicate indirectly, for example via the cell phone, tablet or PC.

In an embodiment, the device can comprise at least one of a user interface and communication means to a computerized device comprising a user interface.

For example, the user interface can be configured for at least one of selecting a desired treatment, indicating the status of the present treatment, indicating an action or reminder to the user, giving warnings, e.g. if the treatment parameters are non-optimal, indicating a target position or target orientation of the device on the subject, and giving information about the statues of the device, such as battery status or cleanliness of the device.

The user interface can be or comprise an acoustic user interface and/or at least one light emitter, such as an LED, configured to give at least one of the indications listed above, for example.

The user interface can be or comprise a display.

The user interface can be arranged on the device body.

In an embodiment, a shape of the contact surface can be adapted to fit or engage with anatomy of the at least one of the subject to be treated and the treatment to be carried out. The shape can be adapted to the subject in the sense that it is adapted to different body portions and/or to different sizes of the same body portion.

In addition, or alternatively, the shape of the contact surface can comprise the indentation designed to be filled by material of the subject in a pressure dependent manner.

In addition, or alternatively, the whole device head can be at least one of adapted to the subject to be stimulated and adapted to the desired stimulation.

In an embodiment, the contact surface can be part of an interchangeable part of the device.

In other words, the device can comprise an interchangeable part that comprises the contact surface.

The interchangeable part can be a portion of the device head or the device head, for example.

In particular, the device can comprise the interchangeable part if the contact surface has a shape adapted to the subject to be stimulated.

If the device head is the interchangeable part, the device head can be at least one of adapted to the subject (body part) to be stimulated and adapted to the desired stimulation (treatment).

The device can comprise a set of interchangeable parts, wherein the parts of the set differ in at least one of the subject to which they are adapted (for example body portion or size of a body portion) and the application to which they are adapted.

The device can comprise means that allow recognition of the interchangeable part attached.

The interchangeable part(s) can be at least one of cleanable, sterile or sterilisable.

In an embodiment, the device comprises a transducer as disclosed with respect to the third aspect.

In other words, the device according to the first aspect is also the device according to the third aspect.

In an embodiment, the device comprises a movable device head as disclosed with respect to the fourth aspect.

In other words, the device according to the first aspect is also the device according to the fourth aspect and—as the case may be—the device according to the third aspect.

A treatment method according to the first aspect comprises:
A step of bringing a device in contact with the subject, wherein the device is configured to apply mechanical energy to the subject by comprising a mass that can oscillate with respect to a housing of the device and the coil, wherein the oscillation is along an axis of the device.
The device can be a device according to any embodiment disclosed. In other words, the method can comprise a step of providing a device according to any embodiment disclosed that is suitable to generate oscillations for the treatment.
A step of detecting a contact between the device and the subject.
A step of setting the mass in oscillation by applying a current to the coil.

In particular, the treatment is a treatment with mechanical energy, in particular with oscillations in a frequency range as given with respect to the device and/or with respect to the transducer.

As mentioned above, a second aspect of the invention concerns a computer-implemented method for supporting a user in a long lasting treatment, said treatment comprising a step of bringing a device, for example a device in any embodiment disclosed above, in contact with a subject to be treated and maintaining the device in this contact before removing the device again.

The device is maintained in this contact for some time, for example for at least 1 second. Usually, the device is maintained in contact to the subject for delivery of mechanical energy, this means for the duration of the treatment, for example.

A method according to the second aspect comprises a step of detecting a contact between the device and the subject and generating an output signal, wherein a characteristic of the output signal is different in case a contact is detected compared to a case in which no contact is detected.

The method comprises further a step of comparing the characteristic of the output signal to a pre-set value.

The contact and its detection, the output signal and its generation, the characteristic of the output signal, its generation and its determination, and the pre-set value can be as disclosed in relation to the first aspect.

In an embodiment, the method comprises a step of measuring a contact pressure between the device and the subject and generating a pressure dependent output signal.

The contact pressure and its measurement, and the pressure dependent output signal and its generation can be as disclosed in relation to the first aspect.

The method can comprise further a step of comparing at least one of the pressure dependent output signal, a characteristic thereof, and the measured contact pressure to a pre-set value.

Comparison of the pressure dependent output signal, of the characteristic or the measured contact pressure to the pre-set value can be as disclosed in relation to the first aspect.

In an embodiment, the method comprises a step of providing a device according to any embodiment and aspect disclosed.

In particular the device provided comprises the sensor element, wherein the contact and—as the case may be—the contact pressure is detected with the sensor element. In the latter case, the sensor element is configured to transform a contact pressure between the contact surface and the subject into a pressure dependent output signal.

The sensor element can be the sensor element according to any embodiment disclosed in relation to the first aspect.

In particular, the sensor element can comprise the capacitive sensor and the indentation that is designed to engage with a body part and be filled by material of the subject in a pressure dependent manner. This also means that the method can comprise a step of filling an indentation by material (e.g. soft tissue such as skin) of the subject in a pressure dependent manner.

In an embodiment, the method comprises a step of determining a treatment quality.

The step of determining a treatment quality comprises a substep of reading out the pressure dependent output signal several times during the time the device is maintained in contact with the subject and a substep of setting the read-out pressure dependent output signals in relation to a pre-set value.

The treatment quality can be determined as disclosed in relation to the first aspect.

The step of determining a treatment quality can be carried out by using a controller and a sensor element configured as disclosed in relation to the first aspect.

The method of determining a treatment quality can comprise a further substep of providing an accordingly configured controller and/or sensor element.

In an embodiment, the method comprises a step of determining a contact quality.

The step of determining a contact quality comprises a substep of determining whether the characteristic of the output signal is greater than the pre-set value. This substep is repeated several times during the time the device is maintained in contact with the subject.

The step of determining a contact quality comprises further a substep of setting the number of determinations having a characteristic that is greater than the pre-set value in relation to the total number of determinations made.

The contact quality can be determined as disclosed in relation to the first aspect.

The step of determining a contact quality can be carried out by using a controller and a sensor element configured as disclosed in relation to the first aspect.

In an embodiment, the method comprises a step of determining treatment completeness.

The step of determining treatment completeness comprises a substep of detecting a start of a treatment and a substep of comparing a number of starts with a pre-set number of treatments.

Treatment completeness can be determined as disclosed in relation to the first aspect.

The step of determining treatment completeness can be carried out by using a controller and a sensor element configured as disclosed in relation to the first aspect.

In an embodiment, the step of determining treatment completeness considers an outcome of at least one of the step of determining a contact quality, the step of determining a treatment quality, and the step of determining treatment regularity.

Consideration of an outcome of at least one of the step of determining a contact quality, the step of determining a treatment quality, and the step of determining treatment regularity can be as disclosed in relation to the first aspect.

At least one treatment parameter can be considered as disclosed in relation to the first aspect in addition or alternatively.

In an embodiment, the method comprises a step of generating an enable signal in case the characteristic of the output signal or—as the case may be—of the pressure dependent output signal is greater than the pre-set value.

The method can comprise a step of activating a transducer automatically after generation of the enable signal.

Generation of an enable signal and activation of the transducer in an automated manner can be as disclosed in relation to the first aspect.

In an embodiment, the method comprises a step of determining a treatment regularity. The step of determining a treatment regularity can comprise a substep of detecting a start of a treatment and a substep of comparing a period between two starts with a pre-set period.

The step of determining a treatment regularity can be as disclosed with respect to the controller being configured to determine a treatment regularity.

The step of determining a treatment regularity can be carried out by using a controller and as disclosed in relation to the device.

A treatment method according to the second aspect is a physical energy treatment method that comprises a computer-implemented method in any embodiment according to the second aspect.

The treatment method comprises further a step of bringing a device, for example a device in any embodiment disclosed, in contact with the subject, wherein the device is configured to apply physical, in particular mechanical, energy to the subject.

In particular, the treatment method can be the treatment for which the computer-implemented method for supporting a user is suitable. This also means, that the treatment method can comprise the step of bringing the device in contact with the subject to be treated and maintaining the device in that contact for some time before removal.

As mentioned above, a third aspect of the invention concerns a device for applying mechanical energy to a subject to be stimulated, wherein the device comprises a transducer that comprises a coil, in particular a coil as disclosed in the following. A coil as disclosed in the following is sometimes called a voice coil.

A device according to the third aspect is suitable for applying mechanical energy, in particular vibration for example in the acoustic energy range or ultrasound, in particular in the low frequency acoustic or even infrasound energy range, to a subject to be stimulated.

In other words, the mechanical energy applied can be oscillations (vibrations) of a specific frequency.

The device may be configured for oscillations of at least about 1 Hz, 5 Hz, 10 Hz, 20 Hz, 30 Hz, 40 Hz, 50 Hz, 60 Hz, 70 Hz, 80 Hz, 90 Hz, or 100 Hz.

The device may be configured for oscillations of at most about 2000 Hz, 1900 Hz, 1800 Hz, 1700 Hz, 1600 Hz, 1500 Hz, 1400 Hz, or 1300 Hz.

The device may be configured for oscillations preferably in the range of 1 Hz to 2000 Hz, more preferably in the range of 20 Hz to 1500 Hz, and more preferably in the range of 60 Hz to 1300 Hz. In other words, the oscillations are preferably in the range of 1 Hz to 2000 Hz, more preferably in the range of 20 Hz to 1500 Hz, and more preferably in the range of 60 Hz to 1300 Hz.

The range of 60 Hz to 1300 Hz can be preferable because the amplitude of an oscillatory motion of a transducer of the kind described below increases with decreasing frequency.

Further, the amplitude and frequency of the oscillatory motion of a transducer of the kind disclosed below can be well controllable in said range. In particular, amplitude and frequency can be better controlled in comparison to alternative transducers.

The device according to the third aspect comprises a device head and optionally a device body. The device body can be designed for being held by a user.

The device can be a handheld device.

The device can be portable.

The device can be configured for a drug free use.

The device can be configured for a non-invasive use.

The device body can be as described with respect to the first and/or fourth aspect.

The device, in particular the device head, is designed to comprise a surface (called “contact surface” in the following) that can be brought in contact to the subject, for example when the device is held at the device body and when the device is in a state suitable for stimulation of the subject.

The device can be configured for direct contact between the surface and the subject, this means between the surface and the skin of the body portion to which the device is applied, during use. In other words, there is no need for an intermediate element or layer between the surface and the skin. In particular, there is no need for a gel and the like.

The device head can be as described with respect to the first and/or fourth aspect.

The device comprises further a transducer configured to convert an electric input signal into an axial movement of a mass. It is the movement of this mass that makes the device suitable for applying mechanical energy to the subject to be stimulated.

In particular, the transducer comprises the mass and is a vibration generator.

The axial movement of the mass can be a movement along a physical axle of the transducer. In other words, the transducer can comprise an axle that is firmly mounted to the housing, this means the axle does not move relative to the housing, but it is an axle of the oscillatory motion of the mass.

In particular, the physical axle is a straight axle.

The axle can define the axis of the device along with the mass oscillates.

The axis can be a longitudinal axis.

The transducer of a device according to the third aspect comprises a coil, in particular a coil as disclosed in the following. A coil as disclosed in the following is sometimes called a voice coil.

In many embodiments, the transducer is arranged in the device head.

The device can be configured such that at least a surface of the device vibrates due to the movement of the mass.

In embodiments, the device can be configured such that the whole device or the device head oscillates, for example.

If it is the device head that oscillates, the device head can oscillate relative to the device body, for example. In other words, the device head can be a vibration unit, wherein the vibration unit is set in vibration by the transducer, in particular by the transducer arranged in the vibration unit.

The mass can be movable mounted relative to a housing of the device, for example a housing of the transducer. The housing of the transducer can be firmly attached to a housing or support of the device, in particular of the device head.

The movement of the mass can be configured to set at least the device head in vibration.

The movement of the mass can be an oscillatory motion, in particular an oscillatory motion along an axis, this means a back and forth movement. The axis can be a normal to the contact surface or a normal of the surface of the subject at the area of contact, for example.

The axis can be defined by the axle.

The oscillatory motion (movement, displacement) can has a frequency as disclosed above in relation to the device according to the third aspect and the device can be configured accordingly.

The device can be configured to sweep over a plurality of frequencies. For example, the device can comprise a controller configured to run the device, in particular the transducer, in a manner comprising a sweep.

For example, the device can be configured to sweep over any frequency range disclosed above in relation to the device according to the third aspect. For example, it can be configured to sweep over the frequency range of 60 to 1300 Hz or a section of it.

Devices configured to sweep over a plurality of frequencies have the advantage that at least one frequency suitable for a specific treatment of a specific (human or animal) individual will be applied. The suitable frequency (or frequencies) for a specific treatment of a specific individual depends on the individual in many cases. Hence, a frequency (or a plurality of frequencies) preset for the specific treatment may not be sufficient for a successful treatment.

There are indications that a frequency suitable for a specific treatment corresponds or is linked to a resonance frequency of the subject, as disclosed in relation to the application example below.

Further, there are indications that a sweep can improve treatment efficiency by exciting a plurality of resonances, also resonances of different kinds as disclosed in relation to the application example below, for example.

Again, the resonance frequencies can be subject-specific. The sweep can also be configured to make sure that at least one resonance frequency is in the applied range of frequencies independent from the stimulated subject.

The sweep over a plurality of frequencies can be characterised by a sweep time, this means by the time needed for scanning from the lowest frequency value of the plurality of frequencies to the largest frequency value and back to the lowest value.

There are indications that a large sweep time, this means the time used to generate the sequence of oscillatory motions having the plurality of frequencies, may have an anti-inflammatory effect.

Further, experiments have shown that a small sweep time improves the energy transfer to the site of application.

The sweep time can be at most about 60 s, 45 s, 30 s, 25 s, 20 s, 15 s, 10 s, or 5 s. The sweep time can be at least about 0.5 s, 1 s, 1.5 s, 2 s, 3 s, 4 s, or 5 s.

The sweep time is preferably between 0.5 s and 30 s, more preferably between 1 s and 10 s.

In an embodiment, the sweep time can vary during a treatment or treatment session. In other words, a sweep rate can vary. In particular, the sweep time can vary during operation within any time range that arise from the sweep times disclosed above. For example the sweep time can vary between 0.5 s and 30 s or between 1 s and 10 s.

For example, the sweep time can decrease during a treatment. In other words, the sweep rate can increase.

A decreasing sweep time (increasing sweep rate) can have the benefit of increased energy transfer at the end of the treatment (treatment session as the case may be). It can further indicate to the user that the end of the treatment (treatment session) is close. The sweep speed can be designed to guide the user through the treatment and make him aware of the status (advancement) of the treatment. For example, he can guess or anticipate from the signal when it will end. In other words, the sweep time can be designed to guide the user through the treatment and make him aware of the status (advancement) of the treatment. For example, the user can guess or anticipate from the signal when the treatment will end.

The device can be configured to carry out a plurality of sweeps during a treatment. In other words, the device can be configured to carry out a plurality of sweeps during the treatment time.

The mass can have a weight of at most about 50 g, 40 g, 30 g, 25 g, 20 g, or 15 g.

The mass can have weight of at least about, 1 g, 2 g, 5 g, or 10 g.

In embodiments, the mass is preferably between 2 g and 20 g.

The weight of the mass can depend on the application and/or subject. In other words, the transducer can be adapted to an application and/or the subject by comprising a mass that is optimized for this application and/or subject in terms of its weight, at least.

An amplitude of the oscillatory motion can be at most 50 mm, 45 mm, 40 mm, 35 mm, 30 mm, 25 mm, 20 mm, 15 mm, 10 mm, 5 mm, or 2 mm.

The amplitude can depend on the application and/or subject. In other words, the amplitude can be adapted to an application and/or the subject. For example, the amplitude can be below 5 mm, in particular below 2 mm for treatments of the paranasal sinuses of a human being.

For example, the amplitude can be 1 mm, 2 mm, 3 mm, 4 mm, or 5 mm.

It has been found that a transducer comprising a coil, in particular a coil as disclosed (and sometimes called a voice coil), can have properties that make such a transducer very suitable for use in the field of mechanical energy therapy in comparison to a piezoelectric transducer or a transducer comprising a rotation mass, for example.

For example, a transducer comprising the coil can generate vibrations that are directed or even focused in a direction. Further—and as pointed out below—the transducer comprising the coil can be designed to have an amplitude of the vibrations that is more homogeneous over the whole frequency range of interest in the field of mechanical energy therapy compared to piezoelectric transducers or a transducer comprising a rotation mass, for example. This is one reason why a transducer comprising the coil can be well suited for the frequencies at the upper end of the frequency range of interest.

As a rule, a transducer comprising the coil and the mass comprises further a permanent magnet or an electromagnet in addition to the voice coil.

In an embodiment, the transducer is adapted to an application of the device in the field of mechanical energy therapy by the mass comprising a permanent magnet, but not the coil.

In other words, it is the coil that is firmly mounted to a housing of the transducer and the magnet that is movably mounted with respect to the housing in this embodiment. Hence, it is the magnet that is actuated and causes the vibration.

This design leads to a heavier mass and allows for higher intensities without increasing space requirements and without increasing the overall weight of the transducer. It further allows for a more homogeneous magnetic field in the actuation region of the transducer without increasing space requirements and without increasing the weight of the transducer. A more homogeneous magnetic field in the actuation region leads to a more linear response of the transducer to the electric input signal and to a more homogeneous amplitude over the frequency range of interest, for example.

In an embodiment, the transducer comprises an axis and the mass is configured to oscillate along this axis.

The axis can be given by the physical axle.

One can envisage various shapes of the permanent magnet, such as ring-, disc-, or square shape.

The permanent magnet can comprise Neodymium, for example. In other words, it can be a so-called Neodymium magnet.

In an embodiment the permanent magnet is a ring magnet, wherein the ring magnet and the coil are arranged concentrically around the axis. For example, the ring magnet and the coil can be arranged concentrically around the axis, wherein the coil is arranged closer to the axis than the ring magnet.

The ring magnet and the coil can be offset along the axis.

The transducer can comprise a plurality (i.e. two or more) of ring magnets. In this case, the ring magnet mentioned before can be considered as a first ring magnet.

The further ring magnet(s) can be arranged concentrically with respect to the axis, too.

The further ring magnet(s) can have the same dimensions as the first ring magnet, and it/they can be offset along the axis. For example, a further ring magnet can be offset along the axis and can be adjacent to the first ring magnet.

The number and arrangement of the further ring magnet(s) can be such that the magnetic field, in particular the magnetic field strength and/or the magnetic field distribution, is optimized with respect to the mass used and/or desired treatment.

In an embodiment, the mass comprises a slit and the slit is concentrically with respect to the axis, too.

In this embodiment the coil can be arranged in the slit. This also means that the slit is or comprises an annular aperture (a ring-shaped opening) that is closer to the axis than the first and—as the case may be—at least one further ring magnet.

In an embodiment, the mass and the ring magnet (or ring magnets) are configured to generate an essentially homogeneous field in a section of the slit, wherein the homogeneous field runs radial to the axis in this section of the slit at least.

For example, the section of the slit in which the essentially homogeneous field is generated can be formed by a portion of the mass forming a core around which the ring magnet (or ring magnets) is arranged and a core ring. The portion of the mass comprising said core is called “core bottom” in the following.

The core ring can be arranged with respect to the ring magnet(s) and the core bottom in a manner that the essentially homogenous field is generated.

The core ring can comprise or can be of a material, in particular of a metal, that is well suited to conduct magnetic fields. In particular, the material can have a high saturation level, for example a saturation level that is larger than 1 T or larger than 1.5 T. The dimensions of the core bottom can be such that a saturation limit of the material (metal) in regards to magnetic field is not exceeded. Thus the core bottom and core ring act in effect as guides for the magnetic flux resulting in the essentially homogeneous magnetic field in the section of the slit.

In an embodiment, an extension of the coil in a direction parallel to the axis, this means a length of the coil, is smaller than an extension of the section comprising the homogeneous field, said extension of the section being in a direction parallel to the axis, too.

In this embodiment, the transducer is configured such that the coil is in the section comprising the homogeneous field independent of the orientation of the transducer.

In particular, it is in the section comprising the homogeneous field in an idle state of the transducer, this means in a state in which no current flows in the coil.

The transducer can further be configured for the oscillatory motion of the mass being restricted between two positions of maximum deflection of the mass and for the coil being predominately in the section of homogenous field.

In particular, the coil can be predominantly in the section of homogeneous field independent of the position of the mass between the two positions of maximum deflection.

A homogeneous magnetic field, in particular in combination with a coil as disclosed that oscillates in the homogenous field only or predominantly is important to have a consistent and well controllable response of the movement of the mass to current generated in the coil.

In an embodiment, the extension of the coil in a direction parallel to the axis is larger than the extension of the section in a direction parallel to the axis.

In this embodiment, the transducer is configured such that a portion of the coil extends over the full extension of the section independent of the position of the mass.

Again, the oscillatory motion of the mass can be restricted between two positions of maximum deflection of the mass.

An embodiment having the coil with an extension that is larger than the related extension of the section has the advantage of a maximum number of windings in the section and independent of the position of the mass, for example. This is advantageous in terms of actuation of the mass, such as actuation force.

In an embodiment, the transducer comprises at least one elastic element that centers the mass when the transducer is not powered.

In particular, the at least one elastic element centers the mass in a manner that the coil is arranged in the slit, in particular in the section of the slit comprising the essentially homogeneous field.

In embodiments, the transducer comprises two elastic elements, for example two elastic elements arranged around or in proximity to the physical axle.

In an embodiment, the at least one elastic element is compressed during oscillation of the mass.

The elastic element or a plurality of elastic elements can be configured to delimit the amplitude of the oscillation.

The elastic element(s) can be configured to delimit a maximal deflection of the mass. In particular, the elastic element(s) can define the two positions of maximum deflection.

Alternatively, the elastic element(s) can be configured such that a stop or a plurality of stops delimit the maximal deflection of the mass. For example, the stop can be given by a bearing of the elastic element(s), such as the housing and/or a coil bracket.

The elastic element can be a spring, in particular a coil spring.

For example, the transducer comprises two elastic elements, wherein one delimits the deflection (amplitude) of the mass in one direction along the axis and the other one delimits the deflection of the mass along the other direction along the axis.

The mass can be suspended by the two elastic elements that may be a coil spring.

The transducer can be configured that no harmonics, in particular no harmonics of significance with respect to the amplitude of the oscillatory motion of the mass at least, are in the frequency range used for the treatment.

This can be done by coordinating the elastic properties of the elastic element and the weight of the mass, for example.

In particular, the transducer can be configured that at least the first (basic) harmonic is outside, in particular below, the frequency range used for treatment.

A transducer that is configured to have no harmonics or at least no harmonics of significance with respect to the amplitude in a determined frequency range is advantageous in combination with devices configured to apply a sweep over a frequency range.

The device can be configured to operate off-resonant. This means that the device can be configured to omit or pass rapidly through frequencies or frequency ranges corresponding to harmonic frequencies.

In an embodiment, the coil is mounted on a support having good heat transfer properties, wherein the support is in thermal connection to a housing of the transducer. The housing is of a material capable to absorb heat generated by the coil and transferred to the housing via the support.

For example, the specific thermal capacity of the housing and/or the support can be larger than 400 J/kg⁻¹ K⁻¹. The housing and/or the support can comprise or consists of steel.

For example, the specific thermal capacity of the housing and/or the support can be larger than 900 J/kg⁻¹ K⁻¹. The housing and/or the support can comprise or consist of aluminium.

In an embodiment, the device can comprise a signal processing unit, wherein the signal processing unit is configured to overlay the electric input signal, this means the input signal used for generating the movement of the mass, with a further signal.

The further signal can be designed to support the treatment caused by the electric input signal. For example, it can be designed to maintain agitated resonance vibrations for a longer period of time.

The further signal can be an audio signal to make the perception of the treatment by the user more pleasant. For example, the further signal can be music or random noise.

In other words, the device can comprise a signal processing unit, wherein the signal processing unit is configured to superimpose a control signal used for the oscillatory motion of the mass with a further signal, wherein the further signal and the transducer (vibration generator) are configured in a manner that an audible signal can be generated from the further signal by the device, in particular by the transducer (vibration generator).

A treatment may be non-pleasant because vibrations that are excited by the device can be conducted to the ear, for example via the bones.

As mentioned above, a fourth aspect of the invention concerns a device for applying mechanical energy to a subject to be stimulated, wherein the device comprises a movable device head that can be moved to a plurality (this means at least two) of positions relative to a device body.

A device according to the fourth aspect is suitable for applying mechanical energy, in particular vibration for example in the acoustic energy range or ultrasound, in particular in the low frequency acoustic or even infrasound energy range, to a subject to be stimulated. In other words, the mechanical energy applied can have any frequency disclosed in relation to the device according to the third aspect. In particular, the frequency can be in the range of 1 Hz to 2000 Hz, for example in the range of 20 Hz to 1500 Hz, preferably in the range of 60 Hz to 1300 Hz.

The device comprises a device body and a device head. The device body can be designed for being held by a user.

The device can be a handheld device.

The device can be portable.

The device can be configured for a drug free use.

The device can be configured for a non-invasive use.

The device body can be as described with respect to the first and/or third aspect.

The device, in particular the device head, is designed to comprise a surface (called “contact surface” in the following) that can be brought in contact to the subject, for example when the device is held at the device body and when the device is in a state suitable for stimulation of the subject.

The device can be configured for direct contact between the surface and the subject, this means between the surface and the skin of the body portion to which the device is applied, during use. In other words, there is no need for an intermediate element or layer between the surface and the skin. In particular, there is no need for a gel and the like.

The device head can be as described with respect to the first and/or third aspect. In particular, it can comprise a sensor element according to the first aspect and a transducer according to the third aspect.

The device head of a device according to the fourth aspect is movable to a first position relative to the device body and to a second position relative to the device body.

The device comprises further a controller configured to switch the device in a sleeping mode if the device head is moved to the first position and to switch the device in an active mode, if the device head is moved to the second position.

In an embodiment, the device head is in addition movable to a third position relative to the device body, wherein the third position allows access to the contact surface for cleaning.

In this embodiment, the controller is configured further to switch the device in the sleeping mode if the device head is moved to the third position.

For example, the device body can comprise a recess and the device head can be designed in a manner that it can be stored completely in the recess. In particular, the device head can be flush with the device body.

In this case, the position of the device head in which it is stored completely in the recess can be the first position.

In this case, the first position can also be considered as a closed position.

A device head being in the closed position is prevented from at least one of contamination, unintentional start and damage, for example.

The device can be equipped for the device head being moved out at least partly of the recess.

For example, the device can comprise an axis around which the device head can be pivoted or along which the device head can moved.

If the device comprises the axis around which the device head can be pivoted and if a rotation angle of 0° corresponds to the first position (closed position, device in sleeping mode), the second position (active mode) can be at a rotation angle between 90° and 150° degrees, for example. For example, the second position can be between 110° and 130°, such as at 115°, 118°, 120°, 122° or 125°.

In particular, the second position can be at most about 150°, 145°, 140°, 135°, or 130°. The second position can be at least about 90°, 95°, 100°, 105°, or 110°.

In such configurations, the optional third position (cleaning mode) can be at a rotation angle between 150° and 200° degrees, for example. For example, the third position can be at 160°, 170°, 180°, or 190°.

In an embodiment, the third position is at 180°.

The device can comprise fixation means that allow automatic or manual fixation of the device head relative to the device body in at least one position.

The device can be configured to move the device head to at least one of the first, second or third position in an automated manner.

Alternatively or in addition, the device can be configured to move the device head between at least two of the first, second and third position in an automated manner.

The device can comprise a motor, in particular an electric drive, configured to move the device head in an automated manner.

Alternatively or in addition to an automated movement of the device head, the device can be configured to move the device head manually.

In an embodiment, the device according to the fourth aspect comprises at least one of a transducer according to any embodiment of the third aspect and a sensor element according any embodiment of the first aspect.

In the sleeping mode and—if present—the cleaning mode, the sensor element can be inactive or even locked. This means also that the sensor element will not generate any output signal that can cause the controller to generate an enable signal. In other words, the device, in particular the controller, prevents a start of a stimulation in this case.

In the sleeping mode and—if present—the cleaning mode, the transducer can be inactive.

The device can be configured to start a stimulation in an automated manner, in particular to activate the transducer, in case the device head is moved to the second position and optionally if the output signal (the pressure dependent output signal as the case may be) is greater than the pre-set value.

As mentioned above, the invention also concerns devices equipped for carrying out the method according to any aspect and any embodiment described in the present text and the methods can comprise any step for operating the device according to any aspect and any embodiment described in the present text.

In particular, at least one of the following can apply in any method disclosed:
Vibrations in the range of 1 Hz to 2000 Hz, for example in the range of 20 Hz to 1500 Hz such as 60 Hz to 1300 Hz can be used to provide the mechanical energy. However, one can envisage to apply vibrations of any frequency disclosed in relation to the device according to the third aspect.
The method can comprise a step of treating the subject with vibrations of a frequency as given above.
A single treatment can be in the range of 2 s to 5 min, in particular between 10 s and 2 min, for example between 30 s and 1.5 min, such as 45 s, 60 s or 75 s. However, one can envisage a treatment time of at least about 0.5 s, 1 s, 2 s, 5 s, 10 s, 15 s, or 20 s and/or a treatment time of at most about 5 min, 4 min, 3 min, 2 min, 90 s, 60 s, 45 s, or 30 s.
In an embodiment, in particular for the treatment of paranasal sinuses, vibrations in the range of 60 Hz to 1300 Hz applied to the cheekbones of a human being and a treatment time of around 1 min per side can be advantageous. These parameters are in particular advantageous if the amplitude at low frequencies is around 2 mm (the amplitude drops with increasing frequency). A sweep as disclosed above can increase the therapeutic efficiency for the treatment of paranasal sinuses further. For example, a sweep having a sweep time of 1 min or 30 s. The latter means that there may be two sweeps during 1 min. A sweep comprising 20 sweeps in 1 min is another example of a sweep that can increase the therapeutic efficiency.
The method can comprise a step of treating the subject during a treatment time given above.
A treatment can comprise a sequence of single treatments, wherein at least two treatments of the sequence of treatments can be carried out at different positions on the subject.
For example, a treatment can comprise an application to the “left” cheekbone followed by an application to the “right” cheekbone.
The method can comprise a step of carry out the treatment a plurality of times. In other words, the method can comprise a plurality of treatment sessions.
The device comprises a sensor element configured to transform a contact and/or a contact pressure between the contact surface and the subject in an output signal and the method can comprise a step of delivering mechanical energy that starts automatically if the output signals is larger than a pre-set value.
The method can comprise a step of switching the device from a sleeping mode to an active mode by moving the position of the device head relative to the device body from a first position to a second position.
The step of putting the contact surface in contact with the subject and the step of delivering mechanical energy can be carried out at least at two different positions on the subject.
For example, these steps can be carried out at two different positions, such as at the cheekbones, in the case of sinusitis treatment. For other treatments, for example migraine treatment, there can be need for more than two positions.
Optionally, the device can indicate the time (moment) to change the position (this means the site of application) on the subject, for example by stopping the transducer.
Optionally, the device or a computerized device can indicate the positions on the subject.

The subject matter of the invention will be explained in more detail in the following text with reference to exemplary embodiments which are illustrated in the attached drawings.

FIG. 1 shows an exterior view of an exemplary embodiment of a mechanical energy therapy device 1, in the following called “device”.

The device 1 shown is a compact, handheld device.

The device comprises a device body 2 and a device head 3, wherein it is in particular the device head 2 that comprises the features related to the invention.

The device head 3 shown comprises a contact surface 4 that is arranged to be brought at least partly in contact with the subject to be treated.

The contact surface 4 may be a surface of an interchangeable part 5 of the device 1

In the embodiment shown, the contact surface 4 comprises an indentation 7 in the shape of a convex recess.

The device head 3 shown is pivoted with respect to the device body 2 (indicated by a double arrow). The pivotal mounting can be such that the device head 3 can be brought at least in the first and second positions relative to the device body 2 mentioned above. In addition, the device head 3 can be optionally brought at least in the third position mentioned above.

In other words, the device head 3 shown is a movable device head.

The device shown comprises further user interface 26 comprising a plurality of LEDs. The LEDs can indicate at least one of a status of the device and a status (advancement) of a treatment or a session of treatments.

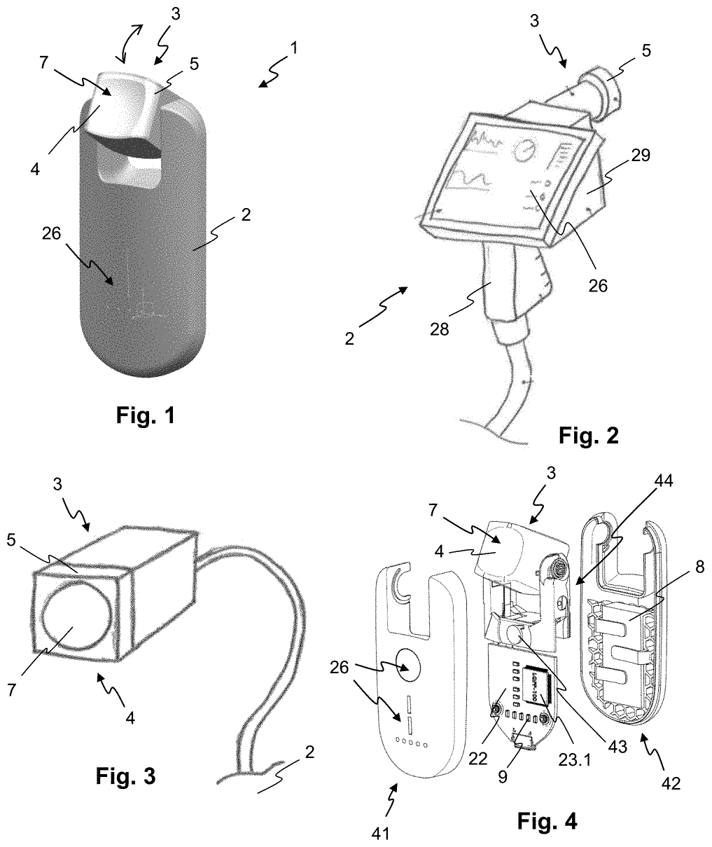

FIG. 2 shows an external view of a further exemplary embodiment of a device 1.

The device 1 shown may be handheld, however it is not as compact as the device 1 of FIG. 1. In other words, the device 1 of FIG. 2 is rather suitable for being installed in or provided by hospitals and professionals whereas the device 1 of FIG. 1 is rather suitable for use by a wider public and can be carried around by a user, for example.

The device 1 of FIG. 2 comprises—in comparison with the device 1 of FIG. 1 at least—a powerful computerized device 29 and a more detailed user interface 26. Optionally, it can comprise a fixture or grip 28.

FIG. 3 shows mainly an external view of an exemplary embodiment of a device head 3.

The device head 3 shown is a handheld device head 3, wherein the device body 2 can be handheld, for example a cell phone or a tablet, or firmly installed, such as a personal computer (PC) or another computerized device, e.g. as shown in FIG. 2.

The device body 2 can supply the device head 3 with power and/or control signals, for example. In the embodiment of FIG. 3, such supply is carried out by a wired connection between device head 3 and device body 2.

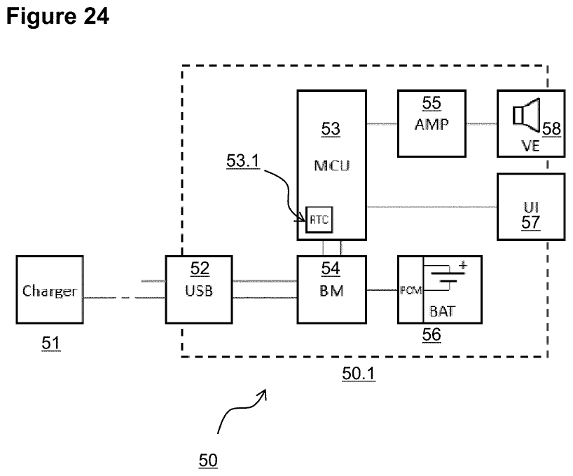

FIG. 4 shows an exploded view of the device 1 shown in FIG. 1. A schematic that shows the corresponding layout of interacting modules is shown in FIG. 24.

In the embodiment shown, a housing of the device body comprises a front part 41 and a rear part 42.

The rear part 42 is equipped to hold a battery 8, for example a rechargeable battery.

Front and rear part are designed to host the more sensitive parts of the device 1, such as a Printed Circuit Board (PCB) 22, a controller 23.1 of the device 1, components of the user interface 26, such as LEDs 9 and at least one manual control element 43 (control knob, button etc.), and at least one support 44 for the device head 3, which is a movable device head in the embodiment shown.

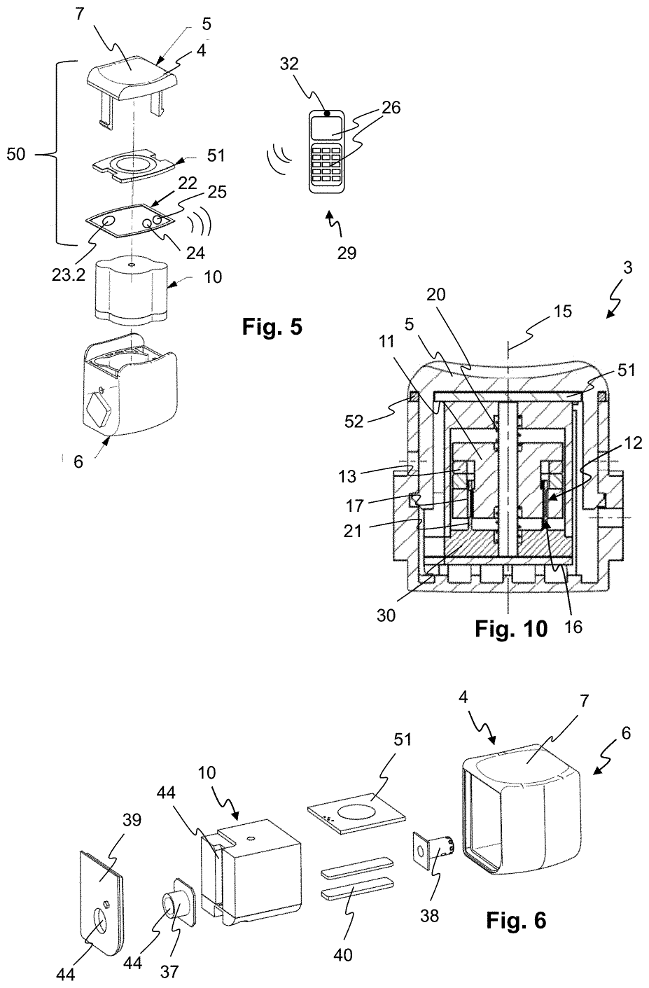

FIG. 5 shows an exploded view of an exemplary embodiment of the device head 3 shown in FIG. 1.

The shape of the device head 3 is given by a housing 6 and the interchangeable part 5.

The interchangeable part 5 can be mounted to the housing 6 by comprising a protrusion arranged on the interchangeable part 5 to reach into the housing 6 and designed to form a positive-fit connection with the housing 6, for example.

The device head 3 shown comprises further a capacitive (touch) sensor 51, a transducer (vibration generator) 10, suitably a voice coil, and a Printed Circuit Board (PCB) 22.

In the embodiment shown, the interchangeable part 5 comprising the contact surface 4 and the indentation 7, the capacitive sensor 51 and the PCB 22 are the main components of a sensor element 50 configured to detect a contact between the contact surface 4 and the subject 100 and to generate a related output signal 45.

The output signal 45 is pressure dependent in some embodiments.

The PCB 22 comprises a controller 23.2 of the sensor element configured to generate the output signal 45.

The PCB 22 can further comprise a memory 24 and/or communication means 25 to a computerized device 29.

In an alternative embodiment, the capacitive sensor 51 comprises the controller 23.2, the memory 24 and/or the communication means 25.

The communication means 25 can be wireless communication means (e.g. Bluetooth or wifi) or wired communication means, as it is the case in the embodiments of FIGS. 2 and 3, for example.

The computerized device 29 can be a handheld (portable, mobile) computerized device, such as a cell phone, laptop or a tablet, or it can be a firmly installed computerized device as disclosed with respect to FIGS. 2 and 3.

The computerized device 29 can comprise a user interface 26 and can be configured to run an application (program or ‘app’) suitable for at least one of controlling the device 1, comparing a characteristic 46 of the output signal 45 with a present value, determining whether the characteristic 46 of the output signal 45 is larger than a pre-set value, generating an enable signal, setting a timestamp when a treatment is started, determining a treatment regularity, determining a treatment completeness, determining a contact quality, determining a treatment quality, and selecting a desired treatment, and indicating the target position and optionally the target orientation, for example.

The controller 23.2 in combination with the memory 24 and/or user interface 26 as the case may be can be configured to carry out one, a plurality or all of the actions listed above. One, a plurality or all of the actions listed above can be carried out by the controller 23.1 of the device 1. In this way the controller may communicate characteristics and treatment parameters to one or more remote computerized devices 29 enabling effective monitoring of treatment.

The controller 23.2 of the sensor element can be integrated in the controller 23.1 of the device 1. The memory 24 and/or the communication means 25 can be arranged on a device PCB 22 as shown in FIG. 4, for example.

FIG. 6 shows an exploded view of a further exemplary embodiment of a device head 3.

In the embodiment shown, the contact surface 4 comprising the indentation 7 is an integral part of the housing 6 of the device head 3.

Due to this, the design of some components of the device head 3 is different compared to the device head 3 according to FIG. 5. For example, the device head 3 comprises a cover plate 39 for closing the device head 3 after arranging the sensor element 50 and the transducer 10 in the housing 6.

The exploded view of FIG. 6 shows further bearing (37, 38) for a pivotal mounting of the device head 2 and ducts 44 for wires. A duct in the cover plate 39, a duct in a bearing 37, and a duct on the transducer 10 is visible in the exemplary embodiment of FIG. 6.

The exploded view of FIG. 6 shows further buffers 40, for example rubber buffers.

The operating principle of the sensor element 50 of FIGS. 5 and 6 is shown in FIGS. 7 to 9.

The operating principle bases on the finding that the indentation 7 leads to a contact surface 4 having a distance from the capacitive sensor 51 that varies and that a filling of the indentation 7 by the subject 100 depends on a contact pressure between the contact surface 4 (and hence the device head 3) and the subject 100, wherein the distance is measured perpendicular to the capacitive sensor 51.

Hence, the sensor element 50 is not only able to detect a contact between the subject 100 and the contact surface 4 (the device 1) and to generate an output signal 45 that differs in the cases of no contact and contact, but also to generate an output signal 45 that is pressure dependent.

FIG. 7 shows the situation when the subject 100 is not in contact with the contact surface 4. An output signal 45 having a characteristic 46, a signal strength in the shown embodiment, of value x is generated.

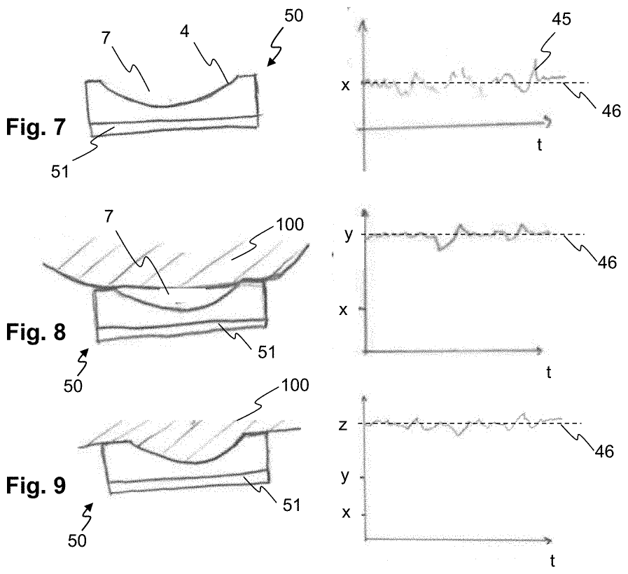

FIG. 8 shows the situation when the subject 100 is in contact with the contact surface 4, but no or only moderate contact pressure is present. An output signal 45 having a characteristic 46, a signal strength in the shown embodiment, of value y is generated.

The maximal distance between the contact surface 4 and the capacitive sensor 51 can be such that a change in capacity induced at the capacity sensor 51 by the subject getting in contact with the contact surface 4 is sufficient to generate detectable offset of the characteristic 46.

FIG. 9 shows the situation when the subject 100 is in contact with the contact surface 4 and a contact pressure sufficient to fill the whole indentation 7 is present. An output signal 45 having a characteristic 46, a signal strength in the shown embodiment, of value z is generated.

The indentation 7 is designed such that an output signal 45 having a characteristic 46, a signal strength in the shown embodiment, between the values y and z is generated in situations in which the subject 100 is in contact with the contact surface 4 and a contact pressure is present, said contact pressure being sufficient to fill the indentation 7 partly only. The exact value if the characteristic 46 depends on the filling state as different filling states (e.g. occupancy or degree of surface area contact) cause different changes in capacity. Hence, the exact value of the characteristic 46 depends on the contact pressure.

It follows from the operating principle that the indentation 7 (the interchangeable part 5 as the case may be) can be subject dependent.

FIG. 10 shows a sectional view of the (assembled) device head 3 of FIG. 5. Among other things, details of the transducer 10 and the positive-fit connection between the interchangeable part 5 and the housing 6 are shown.

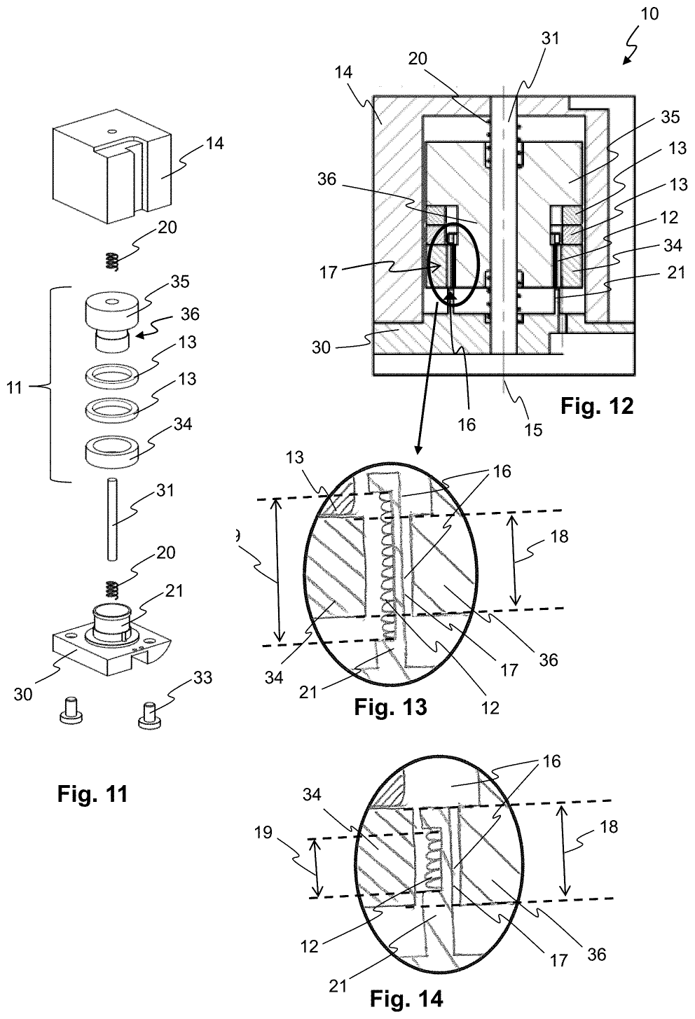

Details of an exemplary embodiment of the transducer 10 are disclosed with respect to FIGS. 11 to 14.

FIG. 10 shows a gasket 52 for sealing an interior of the device head 3 in addition to the components shown in FIGS. 5 and 11-14.

FIG. 11 shows an exploded view of an exemplary embodiment of a transducer 10.

The shape of the transducer is given by a housing 14 of the transducer and a so-called coil bracket 30.

The transducer 10 comprises further a (physical) axle 31 defining a (directional) axis 15, a permanent magnet (two ring magnets 13 in the embodiment shown), a so-called core ring 34, a so-called core bottom 35, and a coil 12 (not shown in FIG. 11), in particular a coil as disclosed in the following. A coil as disclosed in the following is sometimes called a voice coil 12.

The coil bracket 30 can be considered as a base of the transducer 10, said base comprising a support 21 for the coil 12.

The mass 11, this means the component of the transducer 10 that can be actuated to carry out an oscillatory motion along the axis 15, comprises the core bottom 35, the permanent magnet (the ring magnets 13 in the embodiment shown) and the core ring 34.

The core bottom 35 can account for most of the weight of the mass 11. The weight of the core bottom 35 can be adjusted to the application.

The transducer 1 comprises further two elastic elements (coil springs 20) in the embodiment shown. The springs 20 are configured to generate a repelling force to the mass 11.

The elastic elements (coil springs 20) are further configured to position the core ring 34 with respect to the coil 12 when the transducer is not powered.

In the embodiment shown, it is the housing 14 and the coil bracket 30 that delimit the maximum deflections of the mass 11 by the elastic elements (coil springs 20) being partly arranged in a recess of the core bottom and the coil bracket 30, respectively.

FIG. 12 shows a sectional view of the assembled transducer of FIG. 11.

In the embodiment shown, one end of the axle 31 is mounted to the coil bracket 30 and the other end of the axle 31 is mounted to the housing 14.

A first spring 20 is arranged around the axle 31 at its mounting point to the housing 14 and a second spring 20 is arranged around the axle 31 at its mounting point to the coil bracket 30.

The housing 14 and first spring 20 as well as the coil bracket 30 and the second spring 20 define maximal deflections of the mass.

The coil bracket 30 is mounted to the housing 14, for example by screws 33.

In the embodiment shown, the core bottom 35, the ring magnets 13 and the core ring 34 are arranged concentrically with respect to axis 15.

Further, the core bottom 35, the ring magnets 13 and the core ring 34 are firmly mounted to each other, for example by gluing. In other words, the mass 11 is formed integrally (one-piece).

The core bottom 35 comprises a protrusion 36, wherein the ring magnets 13 and the core ring 34 are arranged around the protrusion 36.

The protrusion 36 is designed for forming a slit 16 between the protrusion 36 and the core ring 34. The slit 16 runs concentrically with respect to the axis 15.

The protrusion 36 can be designed further for the slit 16 being formed between the ring magnets 13 and the protrusion 36, too.

The core ring 34 and a portion of the protrusion 36 that forms the slit 16 between the core ring 34 and the protrusion 36 can be designed for an optimized magnetic field in a section 17 of the slit 16 formed by the core ring 34 and the protrusion 36.

The magnetic field is optimized in terms of homogeneity, for example.

In the embodiment shown, the magnetic field lines run (or rather have to run) radial to the axis 15 in said section 17 of the slit 16.

The support 21 and the coil 12 held in position by the support 21 are designed for extending into the slit 16 in a manner that at least a portion of the coil 12 is arranged in the section 17 of the slit 16 formed by the core ring 34 and the protrusion 36. In particular in the idle state of the transducer 10, at least a portion of the coil 12 is in said section 17.

FIG. 13 shows a detail view of the coil 12, the coil ring 34 and the protrusion 36 in the section 17 of optimized magnetic field, this means in an actuation region of the transducer 10.

In the embodiment shown, an extension 18 of the section 17, said extension 18 being parallel to the axis 15, is smaller than a related extension 19 of the coil 12.

In particular, the extension 19 of the coil 12 is such that a portion of the coil 12 extends over the full extension 18 of the section 17 independent of the position of the mass 11.

As shown with respect to FIG. 12, the position of the mass 11 is within two positions of maximal deflection.

A configuration between the coil 12 and the section 17 as shown in FIG. 13 has the advantage of a maximum number of windings being always within the actuation region. This is advantageous in terms of actuation of the mass, such as actuation force.

FIG. 14 shows a detail view of an alternative actuation region.

In the embodiment shown, the extension 18 of the section 17 is larger than the related extension 19 of the coil 12.

In particular, the extension 19 of the coil 12 is such that the whole coil 12 is within the section 17 of optimized magnetic field at least in idle state but independent of the orientation of the transducer 10.

Optionally, the whole coil 12 is within the section 17 of optimized magnetic field independent of the position of the mass 11.

A configuration between the coil 12 and the section 17 as shown in FIG. 14 has the advantage of the coil 12 being in region of homogeneous magnetic field only. This is advantageous in terms of response behaviour of the mass 11 and controllability of the oscillatory motion of the mass, for example.

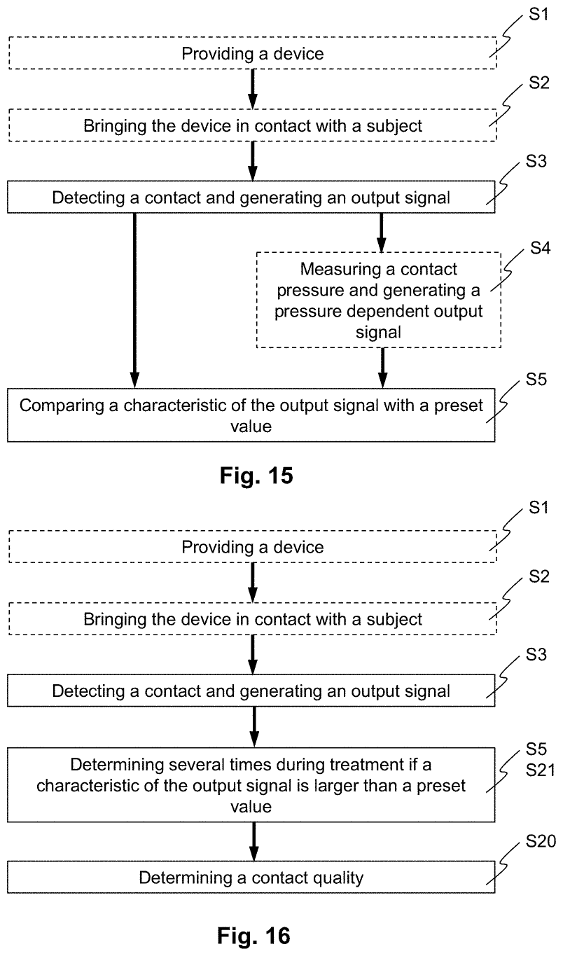

FIGS. 15 to 21 show flow charts of various exemplary embodiments of computer-implemented methods for supporting a user in a mechanical energy treatment. Therein, steps surrounded by a dashed line are optional steps.

FIG. 15 shows the basic steps of an exemplary embodiment of a computer-implemented method for supporting a user in a mechanical energy treatment.

The method comprises a step S3 of detecting a contact and generating an output signal 45 and a step S5 of comparing a characteristic 46 of the output signal 45 with a pre-set value.

The output signal 45, its characteristic 46 and the pre-set value can be as shown with respect to FIGS. 7 to 9, wherein the pre-set value can have the value y.

In embodiments in which not only a contact between the device 1 (in particular its contact surface 4) and the subject 100 is determined but the contact pressure between the device 1 and the subject 100 is determined, the method can comprise the further step S4 of measuring a contact pressure and generating a pressure dependent output signal 45.

The step S4 of measuring a contact pressure and generating a pressure dependent output signal can be considered as a substep of the step S3 of detecting a contact and generating an output signal.

The pressure dependent output signal 45 can be the signal having a characteristic 46 between y and z as discussed with respect to FIGS. 7 to 9.

In such embodiments, the step S5 of comparing a characteristic 46 of the output signal 45 with a pre-set value can comprise comparison of the characteristic 46 between y and z with a pre-set value that is related to the effective contact pressure (for example in Pa=N/m²) between device 1 (contact surface 4) and subject 100.

In particular if the method shown in FIG. 15 is part of a mechanical energy treatment method, the method can comprise further at least one of a step S1 of providing a device, for example a device 1 as shown with respect to FIGS. 1 to 14, a step S2 of bringing the device in contact with a subject 100, and optionally a step of starting and/or carrying out the treatment.

FIG. 16 shows an exemplary embodiment of a computer-implemented method for supporting a user in a mechanical energy treatment, wherein the method comprises a determination of a contact quality.

Compared to FIG. 15, the step S5 of comparing a characteristic 46 of the output signal 45 with a pre-set value is carried out a plurality of times in the method of FIG. 16. Further, the comparison comprises a determination if the characteristic 46 is greater than the pre-set value. In other words, the step S5 of comparing a characteristic 46 of the output signal 45 with a pre-set value corresponds to a step S21 of determining several times during treatment if the characteristic 46 of the output signal 45 is larger than a pre-set value.

The outcome of said step 21 can be used as input for a step 20 of determining a contact quality.

The step 20 of determining a contact quality can comprise a substep of calculating the ration R_{CQ}=N_{P}/N_{T} and a substep of setting the ratio R_{CQ} in relation to a reference value that is representative for a good, enough or insufficient contact quality during treatment as disclosed above.

In particular if the method shown in FIG. 16 is part of a mechanical energy treatment method, the method can comprise further at least one of the step S1 of providing a device, for example a device 1 as shown with respect to FIGS. 1 to 14, the step S2 of bringing the device in contact with a subject 100, and optionally the step of starting and/or carrying out the treatment.

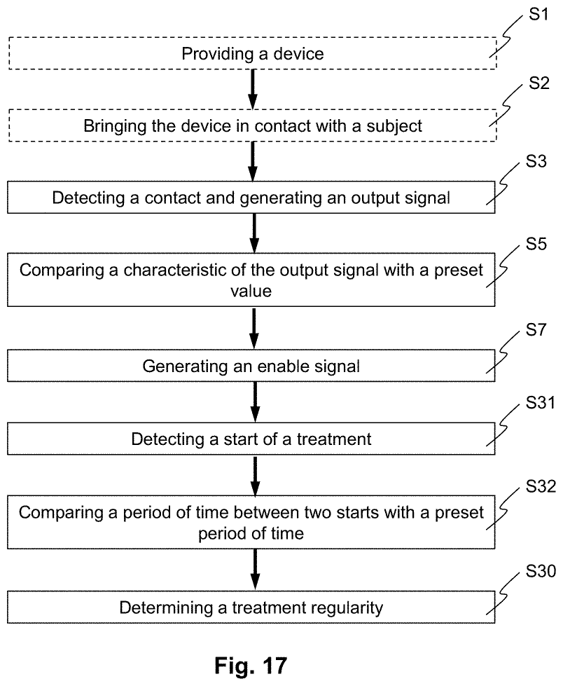

FIG. 17 shows an exemplary embodiment of a computer-implemented method for supporting a user in a mechanical energy treatment, wherein the method comprises a determination of a treatment regularity.

Compared to FIG. 15, the method of FIG. 17 comprises a further step S7 of generating an enable signal if the step S5 of comparing a characteristic 46 of the output signal 45 with a pre-set value has shown that there is a contact, optionally a contact suitable for a treatment, between the device (in particular its contact surface) and the subject.

In the embodiment shown, the method comprises further a step S31 of detecting a start of a treatment, for example by detecting a current applied to the coil 12 of the transducer. The detection of a start can trigger an entry in a memory, said entry comprising the time of the start.

A period elapsed between two starts, and hence between two treatments, can be determined from two entries in a step S32 of comparing a period of time between two starts with a pre-set period of time.

The output of said step 32 or of a plurality of steps 32 can be used to determine a treatment regularity in a step S30 of determining a treatment regularity, for example as disclosed in relation to a controller that is configured to determine a treatment regularity by comparing a period between two timestamps with a pre-set period.

In particular if the method shown in FIG. 17 is part of a mechanical energy treatment method, the method can comprise further at least one of the step S1 of providing a device, for example a device 1 as shown with respect to FIGS. 1 to 14, the step S2 of bringing the device in contact with a subject 100, and optionally the step of starting and/or carrying out the treatment.

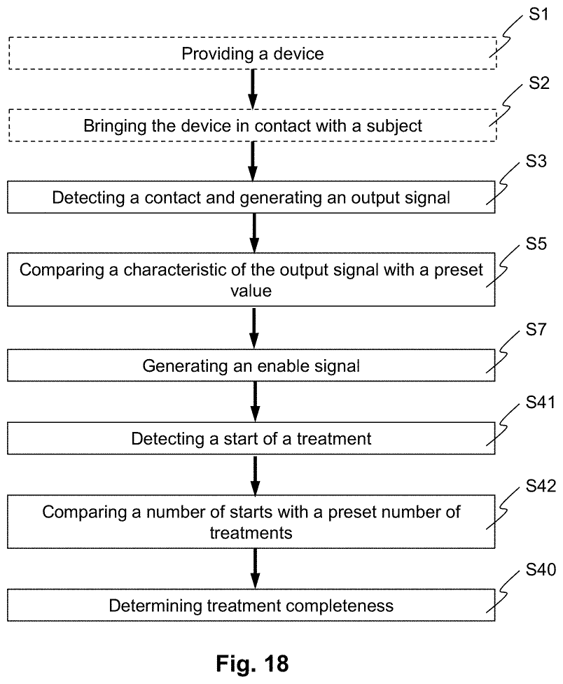

FIG. 18 shows an exemplary embodiment of a computer-implemented method for supporting a user in a mechanical energy treatment, wherein the method comprises a determination of treatment completeness.

Compared to FIG. 15, the method of FIG. 18 comprises the step S7 of generating an enable signal and a step S41 of detecting a start of a treatment, for example by detecting a current applied to the coil 12 of the transducer.

Again, the enable signal can be generated if the step S5 of comparing a characteristic 46 of the output signal 45 with a pre-set value has shown that there is a contact, optionally a contact suitable for a treatment, between the device (in particular its contact surface) and the subject.

The step S41 of detecting a start of a treatment can trigger a counter.

The counter status, i.e. the number of starts detected since the beginning of a treatment, can be used as input for a step S42 of comparing a number of starts with a pre-set number of treatments, said pre-set number can depend on the desired treatment. In particular, it can be the number needed to complete the desired treatment as disclosed with respect to the controller configured to determine a treatment completeness.

The pre-set number of treatments can be a target number of treatments during a pre-determined period of time.

A treatment completeness can be determined from the outcome of the step S42 of comparing a number of starts with a pre-set number of treatments in a step S40 of determining treatment completeness.

In an embodiment, the method comprises a determination of treatment completeness and a determination of treatment regularity. In such an embodiment, the step of detecting a start of a treatment triggers both the counter and the entry in a memory, said entry comprising the time of the start.

In particular if the method shown in FIG. 18 is part of a mechanical energy treatment method, the method can comprise further at least one of the step S1 of providing a device, for example a device 1 as shown with respect to FIGS. 1 to 14, the step S2 of bringing the device in contact with a subject 100, and optionally the step of starting and/or carrying out the treatment.

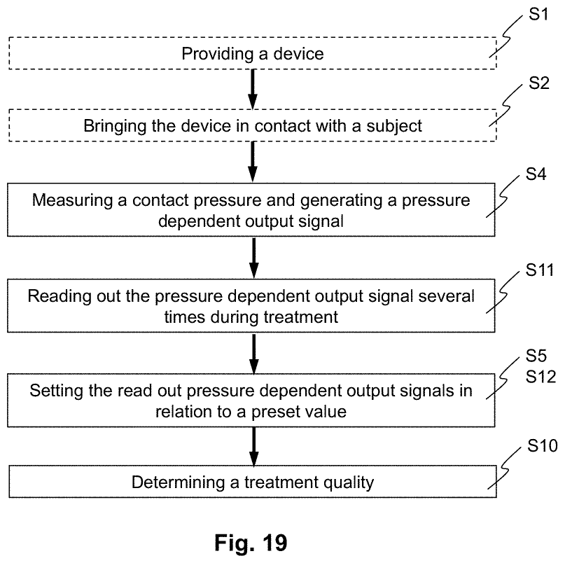

FIG. 19 shows an exemplary embodiment of a computer-implemented method for supporting a user in a mechanical energy treatment, wherein the method comprises a determination of treatment quality.

Compared to FIG. 15, the method of FIG. 19 comprises the optional step S4 of measuring a contact pressure and generating a pressure dependent output signal and it further comprises a step S11 of reading out the pressure dependent output signal a plurality of times during treatment.

Compared to FIG. 15, the step S5 of comparing a characteristic 46 of the output signal 45 with a pre-set value comprises a comparison of the read out pressure dependent output signals with a pre-set value. In other words, the step S5 of comparing a characteristic 46 of the output signal 45 with a pre-set value corresponds to a step S12 of setting the read out pressure dependent output signals in relation to a pre-set value.

The read-out pressure dependent output signals can be processed prior to be set in relation to the pre-set value, for example as disclosed with respect to the controller being configured to read out the pressure dependent output signal several times during a treatment and to determine a treatment quality.

For example, a time evolution of the read out pressure dependent output signals, in particular of the characteristics, can be integrated prior to carrying out the step S12 of setting the (in this embodiment processes) read out pressure dependent output signals in relation to a pre-set value.

The outcome of the step 12 of setting the read out (and optionally processed further) pressure dependent output signals in relation to a pre-set value can be used as input for a step S10 of determining a treatment quality. This can be done as disclosed with respect to the controller being configured to read out the pressure dependent output signal repeatedly during a treatment and to determine a treatment quality, for example.

In particular if the method shown in FIG. 19 is part of a mechanical energy treatment method, the method can comprise further at least one of the step S1 of providing a device, for example a device 1 as shown with respect to FIGS. 1 to 14, the step S2 of bringing the device in contact with a subject 100, and optionally the step of starting and/or carrying out the treatment.

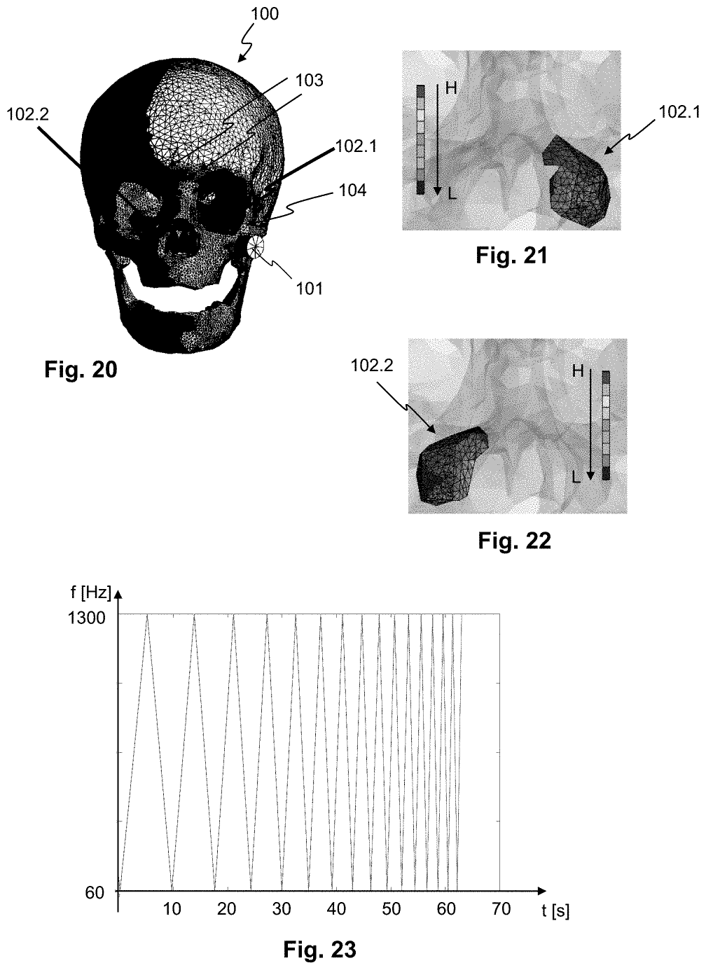

FIGS. 20-23 show an application example of the device, namely the treatment of chronic rhinosinusitis (CRS) by modulated vibration therapy and by use of a device 1 as shown exemplarily in FIGS. 1 and 4 and comprising a transducer 10 as shown exemplarily in FIGS. 11-14.

FIG. 20 shows a model of a human skull. The human skull (more precisely the human head) is the subject 100 in the application example. Such a model of the human skull was used to carry out numerical simulation with the aim to get information about the mechanical, in particular vibrational, properties of the human head and to supply indications of the vibrational excitation of the maxillary sinuses (left maxillary sinus 102.1, right maxillary sinus 102.2) and of the frontal sinuses 103.

The sinuses cannot be seen in FIG. 12 because they are arranged inside the skull (mainly behind maxilla and frontal bone, respectively).

FIG. 21 visualizes a numerically calculated deformation of the left maxillary sinus 102.1 when excited by vibrational energy with a frequency close to a numerically calculated resonant frequency of the maxillary sinus and when the vibrational energy is coupled into the skull by a vibration source at the application point 101, this means by a device in contact with the zygomatic bone 104 at the indicated application point 101.

The colours are indicative for the degree of deformation, wherein the colour next to H indicates a high deformation and the colour next to L indicates a low deformation.

FIG. 22 visualizes a numerically calculated deformation of the right maxillary sinus 102.2 when excited as discussed in relation to FIG. 21. This means, an effect on the right maxillary sinus 102.2 when the vibrational energy is coupled into the left zygomatic bone 104 is shown.

Again, the colours are indicative for the degree of deformation, wherein the colour next to H indicates a high deformation and the colour next to L indicates a low deformation.

FIGS. 21 and 22 show snapshots of the deformation of the maxillary sinuses due to the vibrational energy coupled into the left zygomatic bone 104, only. The time-dependent deformation of the maxillary sinuses is an oscillating deformation between the deformation states shown in FIGS. 13 and 14 and an opposite state.

FIGS. 21 and 22 suggest that the maxillary sinuses can be excited to oscillating deformation by vibrational energy of a specific frequency, i.e. a resonant frequency of the maxillary sinuses, applied to the zygomatic bone 104.

FIGS. 21 and 22 suggest further that a coupling of vibrational energy into the left zygomatic bone may not only have an effect on the left maxillary sinus 102.1 but also on the right maxillary sinus 102.2, and vice versa.

The frequency of the vibrational excitation resulting in FIGS. 21 and 22 was around 355 Hz. However, the numerical simulations suggest various further resonance frequencies between 100 Hz and 1300 Hz, at least.

The numerical simulations carried out supply indications of the structure-mechanical properties of a sinus. Another aspect of the vibrational properties of a sinus can be obtained by approximating the sinus by a Helmholtz resonator and by using the Helmholtz equation to estimate air resonances in the cavity formed by the sinus (by the Helmholtz resonator). A basic resonance frequency of around 27.6 Hz for the sinuses shown in FIGS. 21 and 22 can be calculated from the Helmholtz equation.

Hence, the numerical simulations and the Helmholtz equation suggest that there are resonances of both structure-mechanical and geometrical kind in a frequency range between 20 Hz and 1300 Hz at least, wherein the structure-mechanical resonances can be excited by the device 1 applied to the zygomatic bone 104. Further, it is conceivable that the vibrations applied to the zygomatic bone 104 can excite the resonances of geometrical kind (i.e. the Helmholtz resonances) via deformation of the sinus if the sinus can be approximated by a Helmholtz resonator.

In principle, it is conceivable that an excitation of structure-mechanical and geometric resonances have a synergetic effect, for example by the structure-mechanical resonance(s) opening the ostium of the sinus and enable the appearance of geometric resonance(s).

However, excitation frequencies below 60 Hz are preferably avoided due to possible adverse effects.

Further, literature suggests resonant frequencies of the frontal sinuses between 160 Hz and 1240 Hz.

In summary, a frequency range between 60 Hz and 1300 Hz is a preferred frequency range for the treatment of CRS.

Scanning over a frequency range, for example over the preferred frequency range, guarantees that the sinuses are excited at various resonant frequencies and it guarantees further that subject dependent variations of the resonant frequencies of the sinuses do not have an adverse effect on treatment success.

The influence of sweep time, this means the time for scanning from the lowest frequency value of the preferred frequency range to the largest frequency value and back to the lowest value, on energy transmission from the device 1 to the subject 100 was estimated experimentally. The experiments indicate an increased energy transmission for small sweep times, in particular for sweep times below 5 seconds, whereas the energy transmission is essentially constant for sweep times between 5 and 30 seconds, at least.

In other words, low sweep times seem to be preferable in terms of efficient energy transmission from the device 1 to the subject 100. However, low sweep times are often found unpleasant by the user (patient). Further, the influence of sweep time on the excitation efficiency of the sinuses has to be studied further yet.

Hence, a sweep time that changes during a single treatment seems to be advantageous.

Further, a changing sweep time can be used to make the users perception of the treatment less boring and/or to signal the approaching end of the treatment to the user.

FIG. 23 shows an exemplary course of the vibration frequency produced by the device 1 for CRS treatment. The sweep time decreases from 10 s to 1.5 s. The scanned frequency range is 60 Hz to 1300 Hz.

One can envisage other course of the vibration frequency, for example a course with a constant sweep time and/or sweep time(s) that are within a range given by efficient resonant excitation of a sinus.

A method for treating chronic rhinosinusitis (CRS) by modulated vibration therapy can be as follows when considering the above:
The contact surface 4 of the device 1 is applied to the application point 101 on skin over the left cheekbone of the subject 100.
The device 1 is activated, this means the device generates vibrations in the frequency range between 50 Hz and 1600 Hz, in particular between 60 Hz and 1300 Hz, wherein the frequency range is repeatedly scanned with a sweep time between 0.5 s and 30 s, for example between 1 s and 10 s.
The sweep time can vary during the treatment. For example, the course of the vibration frequency can be as shown in FIG. 23.
The device 1 is deactivated after a pre-set treatment time. The treatment time can be in the range of 0.5 s to 2 minutes, for example 1 minute or 1.5 minute, in the case of CRS treatment.
The treatment is repeated on the right cheekbone.
The treatment time can be longer than the above-disclosed 0.5 s to 2 minutes if the treatment is carried out at one cheekbone, only. In this case, the treatment time can be 2 or 3 minutes or between 2 and 3 minutes, for example.

The method for treating CRS usually comprises a plurality of treatment sessions. This means, the steps listed above are repeated a plurality of times in a given period. In particular, 3 to 4 treatment sessions are carried out per day.

FIG. 24, as mentioned previously provides a schematic of the functional modules that cooperate to form a device of one embodiment of the invention. A device 50 comprises a unitary or modular housing 50.1, comprised within which is a rechargeable battery unit 56. The battery unit 56 comprises a battery cell as well as a battery protection module (PCM). The battery unit 56 is in electrical communication with a power management module 54. An external power supply 51 may be used to charge the device 50 via a communication port 52, such as a USB connection or equivalent. The power management module 54 is in electrical communication with a microcontroller 53 that controls functionality within the device including selection and generation of parameters around vibration frequency ranges and time sweeps. The microcontroller 53 comprises one or more CPU, memory storage and a real time clock 53.1. The microcontroller 53 may further control output from a user interface 57 to provide status, settings, power or error reporting information. The microcontroller 53 may further include communication means allowing telemetry of parameters or other information to remote device via a wired connection—e.g. though the communication port 52—or via wireless communication—e.g. Bluetooth, wifi, or 4G/5G mobile telecommunications. Frequency signal output from the microcontroller 53 is directed to a signal amplifier unit 55 that, in turn, drives a vibration emitter 58, suitably in the form of a voice coil.

### Example

The example relates to design of a randomised, double-blind, multi-centre, clinical trial to assess the safety and efficacy of an innovative vibration therapy portable device for the treatment of chronic rhinosinusitis without nasal polyps (CRSsNP) in adult patients. It will be appreciated that the presently disclosed device is not limited to this specific condition which is identified for exemplary purposes only.

Chronic rhinosinusitis (CRS) is a common disease (e.g. 11% of adults in the UK report symptoms of CRS) leading to substantial economic burden. The symptoms include nasal obstruction, nasal discharge, facial pain, loss of smell and sleep disturbance and have a major impact on patient's quality of life. Acute exacerbations, inadequate symptom control and respiratory disease exacerbation are common, which is in part due to considerable variation in the way CRS is managed. Currently, two main clinical forms are distinguished: CRS with polyps (CRSwNP), which are hyperplastic swellings of the nasal mucosa, and CRS without nasal polyps (CRSsNP). CRSwNP accounts for about a third of all recorded CRS cases and often requires surgical intervention. Intranasal steroids are frequently used to treat CRSsNP. However, the accepted treatment with topic corticosteroids and nasal irrigation and antibiotics as needed is insufficient for many patients suffering from CRS. For the fact that there is no common agreed standard of care for CRSsNP the present inventors defined according to the EPOS guidelines treatment with relevance and evidence level A as standard of care (Fokkens W J, et al. (2012) “European Position Paper on Rhinosinusitis and Nasal Polyps”. Rhinol Suppl.: 2012 March (23): 1-298). Hence, there is a need for alternative or adjunct therapeutic options to fill the gap between the medical and surgical options of treatment.

In this study rationale, a non-invasive, comfortable and easy to apply portable device can be used. In order to allow use by lay persons, the device does not require maintenance or change of any parts, can easily be recharged and complies with aesthetic and privacy concerns.

Device Description, Components and Materials

The device for use in the trial is a portable handheld medical device, of the type shown in FIGS. 1, 4 and/or 24 described above, for patient-use at home that sends vibrations to the paranasal sinuses via the cheekbone (e.g. see location 101 in FIG. 20). The device is indicated for treatment of CRSsNP in adults. It is meant for unattended use by the patient at home. The intended part of body/tissue in contact is the skin of the cheeks. A key component for the performance of the device used in this trial is the presence a controllable vibration emitter in the form of a voice coil.

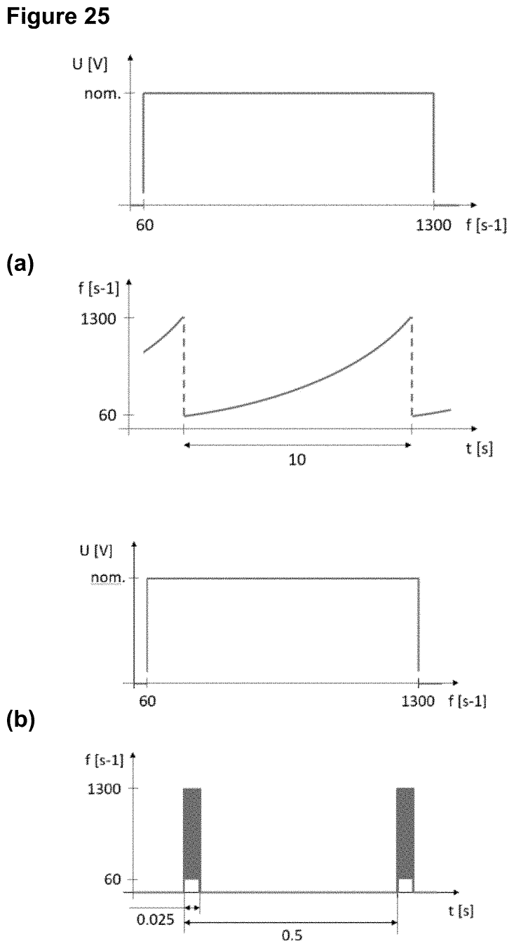

The acoustic signal being the relevant output of the device, its two constituents, amplitude (i.e. volume) and sweep, are schematically shown in FIG. 25(a) in the frequency and time domain for the therapeutic study device.

For the sake of blinding, a comparator or control device is by appearance identical to the study device but produces only an intermittent acoustic noise for 5% of the treatment duration, with the aim of producing minimal resonance in the paranasal sinuses. The signal of the control device is shown in FIG. 25(b).

Upon loading of the firmware, each device is randomly programmed either as a study device or a control device.

Mechanism of Action

The device is intended to stimulate the mucus flow from the maxillary sinus and promote sinus ventilation by vibration via the cheekbone. The proposed mode of action and rationale for the test device is based on principals of promotion of sinus ventilation and mucus flow, reduction of inflammation and CRS related pain.

Promotion of Sinus Ventilation and Mucus Flow

A reduced NO level in the nasal airflow is often used as indirect measurement to determine the ventilation of and mucus retention in the paranasal sinuses (Arnal, J. F, et al. (1999 Eur Respir J 13(2): 307-312). Vibration therapy with the device aims at the creation of an oscillating airflow between the sinus and the nasal cavity, thus promoting sinus ventilation and the drainage of accumulated mucous and inflammatory secretions. In order to create an oscillating airflow, the vibrations sent to the maxillary sinus should be at its resonance frequency, since vibrations applied at the resonance frequency of the maxillary sinus lead to sinus ventilation (as measured by a sudden increase in nasal NO exhalation).

Reduction of Inflammation and Analgesia

Whole-body vibration (WBV) therapy has gained popularity for various indications, including inflammatory diseases like chronic obstructive pulmonary disease (COPD), fibromyalgia, or osteoarthritis, due to its suggested anti-inflammatory effect. Further, the clearance of mucous and secretions from the respiratory system by the application of high frequency chest wall oscillations (HFCWO) can reduce plasma levels of C-reactive protein and the number of inflammatory cells in sputum samples. Vibration is also used for pain relief before administration of local anaesthetics at the dentist (DentalVibe®) or for oral-facial pain.

The following considerations relate to the selected frequency and instructed application pressure:

Frequency

The vibration frequency of the device should be at the resonance frequency of the paranasal sinus to support sinus ventilation. In 10 healthy adults, measurement of the maxillary sinus size by computer tomography revealed a large variation of 4-22 cm³. The resulting estimated resonant frequencies (based on Helmholtz theory) were ˜110-350 Hz (Tarhan, E., et al. (2005). J Appl Physiol (1985) 99(2): 616-623). However, due to the variability in anatomy and mucus retention between patients a broader frequency range from 60-1300 Hz may be selected.

Directions for Use in Trial

The device shall be used three times a day, such as in the morning, in the afternoon and before bedtime for one minute each side of the face applied respectively to the right and left cheekbone.

Primary Clinical Endpoint

The primary endpoint is the change in subjective symptoms as quantified by the German validated disease-specific 20-item Sino-nasal Outcome Test (SNOT-20 GAV) after 12 weeks. Superiority is defined as more than minimal clinically important difference (MCID) of 8.9 points to active control of SNOT-20 score at 12 weeks. A range of secondary endpoints may also be considered including a reduction in the need for systemic medication (e.g. antibiotics or steroids), reduction or avoidance of surgical intervention, and reduction in pain or discomfort.

Results of Small Scale Prototype Testing

A prototype device has been tested to evaluate muco-ciliary clearance time using the saccharine transit time test (Andersen I, et al. (1974) Arch Environ Health; 29 (05) 290-293) and sinuses ventilation (exhaled nNO). The results consistently indicate a) increasing speed up of the muco-ciliary transport (4 times faster under saccharine transit time test), and b) an average 7-fold increase (1387 ppb vs 198 ppb) of exhaled nNO within the first few seconds of applying the vibration to the subject.

The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims.

1. A therapeutic device for applying mechanical vibrational energy stimulation to a subject, wherein the device comprises a housing and the housing comprises:
i. a contact surface for being put in contact with the subject;
ii. a sensor element configured to detect a contact between the contact surface and the subject and optionally to transform a contact pressure between the contact surface of the device and the subject to which the mechanical vibrational energy is to be applied into a pressure dependent output signal; and
iii. a transducer configured to convert an electric input signal into an axial oscillatory motion of a mass, wherein the transducer comprises a coil and a permanent magnet, wherein the mass can be moved relative to the housing, wherein the relative movement of the mass is configured to cause at least the contact surface to vibrate, and wherein the mass comprises the permanent magnet.

2. The device of claim 1, wherein a characteristic of the output signal is different in case contact with the subject is detected compared to when no contact is detected.

3. The device of claim 1, wherein the sensor element comprises a capacitive sensor.

4. The device of claim 3, wherein the capacitive sensor is configured to detect the subject when in contact with the contact surface.

5. The device of claim 1, wherein the contact surface comprises at least one indentation.

6. The device of claim 5, wherein the at least one indentation is arranged relative to the capacitive sensor such that different filling states of the indentation lead to different pressure dependent output signals of the sensor element.

7. The device of claim 1, further comprising a controller, wherein the controller is configured to determine whether the characteristic of the output signal is greater than a pre-set value.

8. The device of claim 7, wherein the device is configured to prevent a start of a stimulation if the characteristic of the output signal is below the pre-set value.

9. The device of claim 8, wherein the pre-set value corresponds to a minimum threshold contact pressure between contact surface and the subject.

10. The device of claim 7, wherein the controller is configured to set a timestamp when a stimulation is started.

11. The device of claim 10, wherein the controller is configured to determine a treatment regularity by comparing a period between two timestamps with a pre-set period.

12. The device of claim 10, wherein the controller is configured to determine a treatment completeness by comparing a number of timestamps with a pre-set number of treatments.

13. The device of claim 7, wherein the controller is configured to determine whether the characteristic of the output signal is greater than the pre-set value repeatedly during a treatment and to determine a contact quality by setting the number of characteristics greater than the pre-set value in relation to the total number of output signals.

14. The device of claim 1, further comprising at least one of a user interface and communication means to a computerized device comprising a user interface.

15. The device of claim 1, wherein a shape of the contact surface is adapted to fit or engage with the anatomy of the subject to be stimulated and the treatment to be carried out.

16. The device of claim 15, wherein the contact surface is comprised within an interchangeable part of the device.

17. The device of claim 1, wherein the housing comprises a device body and a device head, and wherein the device head is movable to a first position relative to the device body and to a second position relative to the device body, and wherein the contact surface is located on the device head.

18. The device of claim 17, wherein the device comprises a controller configured to switch the device in a sleeping mode if the device head is moved to the first position and to switch the device in an active mode, if the device head is moved to the second position.

19. The device of claim 18, wherein the device head is movable to a third position relative to the device body, wherein the third position allows access to the contact surface for cleaning and wherein the controller is configured to switch the device in the sleeping mode if the device head is moved to the third position.

20. The device of claim 1, wherein the transducer comprises an elastic element that centers the mass when the transducer is not powered.

21. The device of claim 20, wherein the elastic element is compressed during operation of the transducer.

22. The device of claim 1, wherein the transducer is configured to oscillate at a frequency of not less than 1 Hz, 5 Hz, 10 Hz, 20 Hz, 30 Hz, 40 Hz, 50 Hz, 60 Hz, 70 Hz, 80 Hz, 90 Hz, or 100 Hz.

23. The device of claim 1, wherein the transducer is configured to oscillate at a frequency of not more than about 2000 Hz, 1900 Hz, 1800 Hz, 1700 Hz, 1600 Hz, 1500 Hz, 1400 Hz, or 1300 Hz.

24. The device of claim 1, wherein the transducer is configured for oscillations in the range of 1 Hz to 2000 Hz.

25. The device of claim 1, wherein the transducer is configured to sweep over a frequency range of about 60 to about 1300 Hz, or a section thereof.

26. The device of claim 25, wherein the sweep occurs over a time period of at most about 60 s, 45 s, 30 s, 25 s, 20 s, 15 s, 10 s, or 5 s.

27-39. (canceled)
